(19) United States
(12) Patent Application Publication
(10) Pub. No.: US 2007/0065829 A1
(43) Pub. Date: Mar. 22, 2007

(54) In vitro differentiation of vascular smooth muscle cells, methods and reagents related thereto

(75) Inventors: Mu-En Lee, Newton, MA (US); Mukesh Jain, W. Newton, MA (US); Masafumi Watanabe, Brookline, MA (US)

Correspondence Address: FOLEY HOAG, LLP;PATENT GROUP (w/HUV HMV), 155 SEAPORT BLVD., BOSTON, MA, 02210-2600, US

(73) Assignee: President and Fellows of Harvard College, Cambridge, MA (US)

(21) Appl. No.: 10/693,316

(22) Filed: Oct. 23, 2003

Related U.S. Application Data
(63) Continuation of application No. 09/181,311, filed on Oct. 28, 1998, now abandoned.
(60) Provisional application No. 60/063,363, filed on Oct. 28, 1997.
(60) Provisional application No. 60/080,420, filed on Apr. 2, 1998.
(60) Provisional application No. 60/096,685, filed on Aug. 14, 1998.

(51) Int. Cl. C12Q 1/68; C12N 5/08

(52) U.S. Cl. 435/6; 435/366

(57) ABSTRACT

This invention is directed to an in vitro system for rapidly and uniformly inducing immortalized neural crest cells to differentiate to vascular smooth muscle cells. As excessive proliferation of vascular smooth muscle cells is a phenotypic response to the development of occlusive arteriosclerotic disease, the in vitro system of this invention is used to identify molecular regulators of smooth muscle cell development and differentiation. As the molecular regulators of smooth muscle cell differentiation are identified, the invention also encompasses methods to isolate the genes coding for these regulators. This invention also relates to molecules identified through the use of the invention's in vitro system, as well as to compounds that inhibit or regulate the identified molecules.

### GOVERNMENT SUPPORT

Work described herein was supported under grants awarded by the National Institutes of Health. The U.S. government therefore may have certain rights in this invention.

### FIELD OF THE INVENTION

This invention is in the cardiovascular field, directed to an in vitro system for rapidly and uniformly inducing immortalized neural crest cells to differentiate to vascular smooth muscle cells. As excessive proliferation of vascular smooth muscle cells is a phenotypic response to the development of occlusive arteriosclerotic disease, the in vitro system of this invention is used to identify molecular regulators of smooth muscle cell development and differentiation. As the molecular regulators of smooth muscle cell differentiation are identified, the invention also encompasses methods to isolate the genes coding for these regulators. This invention also relates to molecules identified through the use of the invention's in vitro system, as well as to compounds that inhibit or regulate the identified molecules.

### BACKGROUND OF THE INVENTION

Arteriosclerosis generally describes the thickening and hardening of the arterial wall. Arteriosclerosis and its complications, such as heart attack and stroke, are the major causes of death in developed and developing countries. (Ross (1993) Nature 362:801-809.)

Vascular smooth muscle cells constitute the major portion of the blood vessel wall. Differentiated vascular smooth muscle cells function mainly to regulate vascular tone. Fully differentiated vascular smooth muscle cells proliferate at an extremely low rate, do not migrate, and do not synthesize extracellular matrix. Vascular smooth muscle cells also express unique contractile proteins, ion channels, and signaling molecules required for their contractile function. In contrast to skeletal and cardiac muscle cells, vascular smooth muscle cells are not terminally differentiated in adult animals.

In response to injury to the blood vessel wall, such as by hypercholesterolemia, hyperhomocystenemia, hypertension, or trauma, vascular smooth muscle cells dedifferentiate and assure a proliferative, migratory, and synthetic phenotype. Phenotypic changes in vascular smooth muscle cells are a hallmark of occlusive arteriosclerotic diseases. (Ross (1993) Nature 362, 801-809; Owens (1995) Physiol. Rev. 75, 487-517; Schwartz, et al. (1990) Physiol. Rev. 70, 1177-1209.) Nonetheless, despite the importance of phenotypic alterations of vascular smooth muscle cells, little is known about the molecular mechanisms regulating differentiation of this cell type.

The identification of molecular regulators of smooth muscle cell differentiation is essential to the design of strategies for treating vascular disease. Research into molecular mechanisms regulating smooth muscle cell differentiation has been hindered by the lack of an in vitro cell differentiation system.

### SUMMARY OF THE INVENTION

This invention is directed to an in vitro system for rapidly and uniformly inducing immortalized neural crest cells to smooth muscle cell differentiation. This neural crest cell to smooth muscle cell model facilitates the identification of nodal regulators of smooth muscle development and differentiation. Thus, this invention is also directed to molecules that are identified using this in vitro system. This invention is further directed to the compounds that inhibit or regulate the nodal regulators identified from this in vitro system. Methods to isolate the genes coding for these nodal regulators also fall within the scope of the invention. Thus, smooth muscle cell differentiation can be maintained with agents that inactivate nodal regulators of smooth muscle cell dedifferentiation.

This invention is also directed to methods for treating or preventing arteriosclerosis by inhibiting or regulating the activity of smooth muscle cell differentiation by administration of the compounds that inhibit or regulate the nodal regulators identified from use of this in vitro system. Such compounds can be used in the treatment of vascular trauma is caused by organ transplant, vascular surgery, transcatheter vascular therapy, vascular grafting or placement of a vascular shunt or intravascular stent. They may also be used in the treatment of vascular and cardiovascular indications characterised by decreased lumen diameter, e.g., coronary heart disease, smooth muscle neoplasms, uterine fibroid, obliterative diseases of vascular grafts and transplanted organs and other vascular smooth muscle and endothelial cell proliferative disorders.

Modulation of smooth muscle cell differentiation mediated by the molecular regulators identified through the in vitro smooth muscle cell differentiation system of this invention may be effected by agonists or antagonists of molecular regulators as well. Screening of peptide libraries, compound libraries, and other database in the gene banks to identify agonists or antagonists of the function of molecular regulators is accomplished with assays for detecting the ability of potential agonists or antagonists to inhibit smooth muscle cell dedifferentiation.

For example, high through-put screening assays may be used to identify compounds that modulate the differentiation activity of the smooth muscle cell. These screening assays facilitate the identification of compounds that inhibit smooth muscle cell dedifferentiation. For example, an in vitro screen for compounds that disrupt the molecular regulators activity comprises multiwell plates of the in vitro smooth muscle cell differentiation system, and, after a sufficient time after differentiation, incubating the system in the presence of one or more compounds to be tested. Molecules that specifically disrupt the interaction could, in principle, bind to either the molecular regulator or interfere with molecular regulator receptor. Either class of compound would be a candidate smooth muscle cell modulating agent.

### DESCRIPTION OF THE DRAWINGS

FIG. 1A shows undifferentiated neural crest Monc-1 cells (controls). FIG. 1B shows the morphologic changes after differentiation of Monc-1 cells into smooth muscle cells after 4 days of incubation in smooth muscle cell differentiation medium (SMDM). The original magnification for both pictophotographs was 200×.

FIG. 2 shows the effect of exogenously added active TGF-β1 has on smooth muscle cell differentiation. Total RNA (10 μg) was analyzed from cultured Monc-1 cells in SMDM (lane 1), complete medium (lane 2), and SMDM supplemented with activated TGF-β1 (10 ng/ml) (lane 3) for 33 hours. The gels were hybridized with a ³²P-labeled fragment of SM22α.

FIG. 3 shows mRNA analysis of smooth muscle markers after differentiation of Monc-1 cells down the smooth muscle and neuronal lineages. Total mRNA was harvested 0, 2, 5, and 8 days after incubation in SMDM and probed for the transcripts indicated. The same blot was also hybridized with an 18S oligonucleotide probe to confirm equivalent loading. Expression of all the genes tested in the Monc-1 cell to smooth muscle cell differentiation system was similar to that in the aorta, where smooth muscle cells are highly differentiated, as shown in FIG. 3.

FIG. 4 shows the expression of smooth muscle myosin heavy chain isoform SM1 in differentiated Monc-1 cells. In FIG. 4A, total RNA was harvested from stripped mouse aortas and Monc-1 cells at 0 (Undifferentiated) and 6 (Differentiated) days after placement in SMDM. Reverse transcription PCR was performed with primers designed from the mouse SM1 and SM2 cDNAs. To control for efficiency of reverse transcription, an aliquot was analyzed for glyceraldehyde-3-phosphate dehydrogenase (GAPDH). In FIG. 4B, total cellular proteins isolated from stripped mouse aortas (10 μg) and Monc-1 cells (25 μg) treated as in FIG. 4A were analyzed by Western blotting with anti-smooth muscle myosin heavy chain antibody. The asterisk denotes non-muscle myosin heavy chain.

FIG. 5A shows the induction of SM22α promoter after differentiation. Monc-1 cells were transfected by electroporation with 12.5 μg of the indicated expression plasmid and 2.5 μg of CMV-βGAL to control for transfection efficiency. Cell extracts were assayed after 72 hours in complete medium (Ctrl) or SMDM (Diff) (means±SE, n=6, *p<0.05). FIG. 5B shows the electrophoretic mobility shift analysis with the CArG element of the SM22α promoter in nuclear extracts from undifferentiated Monc-1 cells (Control) and cells incubated in SMDM (Differentiated) for 8 days. A 250-fold excess of identical (I) and nonidentical (NI) oligonucleotides was applied. Supershift analysis was done with antibodies to serum responsive factor (SRF) and YY1.

FIG. 6 shows the nucleic acid sequence homology between the 153 base pair fragment W011 and the rat LTBP-1.

FIG. 7 shows a Northern blot of RNA that was isolated from growing rat aorta smooth muscle cells (RaSMC) (lane 1) and confluent RaSMC (lane 2). Total RNA (10 μg) was transferred to a membrane and hybridized with a ³²P-labeled W011 fragment.

FIG. 8 shows a Northern blot of RNA that was isolated from undifferentiated Monc-1 cells (lane 1), Monc-1 cells cultured in SMDM for 3 (lane 2), 11 (lane 3), 24 (lane 4), and 120 (lane 5) hours, and Monc-1 cells cultured in neuronal differentiation medium for 24 (lane 6) and 96 (lane 7) hours. Total RNA (10 μg) was transferred to a membrane and hybridized with a ³²P-labeled W011 fragment.

FIG. 9A shows the deduced amino acid sequences of human and mouse ACLP. A, deduced open reading frames of human ACLP and mouse ACLP. The human and mouse proteins contain 1158 and 1128 amino acids, respectively. Bullet marks initiating methionine in mouse AEBP1. Highlighted motifs include a signal peptide (boldface, underlined), a 4-fold lysine- and proline-rich repeating motif (boldface italic), a discoidin-like domain (boldface italic, underlined), and a region with homology to the carboxypeptidases (boldface).

FIG. 9B is a schematic representation of human ACLP. Marked are the signal peptide sequence at the N terminus (Signal), the 4-fold repeating motif (Repeat), the discoidin-like domain (DLD), and the region with homology to the carboxypeptidases (CLD).

FIGS. 10A and B show in vitro transcription and translation of mouse ACLP and identification of ACLP. FIG. 10A, the mouse ACLP cDNA in pCR2.1 was transcribed and translated in vitro in the presence of [35S]methionine. An aliquot of this reaction was resolved on a 6% sodium dodecyl sulfate-polyacrylamide gel that was then dried and exposed to film at room temperature. FIG. 10B, Western blot analysis of proteins extracted from MASMCs. After total cellular protein lysates had been prepared as described under “Experimental Procedures,” 50-μg aliquots were resolved on a 6% sodium dodecyl sulfate-polyacrylamide gel. The gel was transferred to a nitrocellulose membrane and incubated with a polyclonal anti-ACLP primary antiserum and a horseradish peroxidase-conjugated goat anti-rabbit secondary antibody. The blot was visualized by enhanced chemiluminescence and exposed to film at room temperature.

FIGS. 11A, B, C and D. Cellular localization of mouse ACLP. RASMCs and A7r5 cells were transfected transiently with an ACLP expression construct tagged with c-myc at the C terminus. Fusion protein was detected with an anti-c-myc antibody (A and C), and nuclear DNA was counterstained with Hoechst 33258 (B and D). RASMCs and A7r5 cells both exhibited strong perinuclear staining that was excluded from the nucleus, as demonstrated by nuclear DNA counterstaining. Initial magnification, ×400.

FIGS. 12A and B. ACLP mRNA and protein expression in mouse tissue. A, total RNA was isolated from mouse tissues as described under “Experimental Procedures,” and 10-μg aliquots were resolved on an agarose-formaldehyde gel, transferred to a nitrocellulose filter, and hybridized to a 32P-labeled fragment of mouse ACLP. Equal loading was verified by hybridization to a 32P-labeled oligonucleotide complementary to the 18S ribosomal RNA. B, total cellular protein was extracted from mouse organs, and 50-μg aliquots were subjected to Western blotting with a polyclonal anti-ACLP antiserum, which was detected with horseradish peroxidase-conjugated secondary antibody and enhanced chemiluminescence.

FIGS. 13A, B C and D. Detection of ACLP mRNA in aorta by in situ hybridization. Rats were perfused with 4% paraformaldehyde, and tissue was removed and sectioned as described under “Experimental Procedures.” Sections from aorta (A and B) and skeletal muscle (C and D) were hybridized with [35S]UTP-labeled antisense (A and C) or sense (B and D) riboprobes. Magnification, ×600.

FIGS. 14A and B. Increase in ACLP mRNA and protein levels in serum-starved aortic smooth muscle cells. A, total cellular RNA was extracted from MASMCs and RASMCs cultured in 10% fetal bovine serum (Growing) or 0.4% calf serum (Quiescent) for 3 days. RNA was fractionated on a 1.2% agarose-formaldehyde gel, which was transferred to a nitrocellulose filter, hybridized to a 32P-labeled fragment of mouse ACLP, and normalized to 18S rRNA. B, MASMCs treated as in A were harvested for total cellular protein and immunoblotted as described in the legend to FIG. 10B.

FIGS. 15A and B. Induction of ACLP mRNA and protein as Monc-1 cells differentiate into smooth muscle cells. A, RNA was extracted from Monc-1 cells cultured on fibronectin before induction of differentiation (0) or 1, 2, 4, and 6 days after treatment with differentiation medium. RNA blots were hybridized to mouse ACLP and smooth muscle-actin (Sm-actin) cDNA probes labeled with 32P. Equal loading was verified by hybridization to a ³²P-labeled oligonucleotide complementary to the 18S ribosomal RNA. B, protein extracts were prepared from Monc-1 cells harvested before differentiation (0) or 6 days after differentiation into smooth muscle cells. Extracts were examined for ACLP expression as described in the legend to FIG. 10B. MASMCs were used as a positive control.

FIGS. 16A and 16B are pictures of gels showing the expression pattern of certain differentially displayed genes.

FIG. 17 shows the expression pattern of TGFβ1, LTBP-1 and Decorin.

### DETAILED DESCRIPTION OF THE INVENTION

Vascular smooth muscle cells (VSMCs) are the predominant component of the blood vessel wall, where their principal function is to regulate vascular tone. Although VSMCs normally exist in a differentiated state, they can dedifferentiate and proliferate in response to certain stimuli. Activation of VSMCs from a contractile and quiescent state to a proliferative and synthetic state contributes to several disease processes, including arteriosclerosis. Defining effectors that modulate VSMC function and identifying marker proteins that characterize a given VSMC phenotypic state will contribute to our understanding of the mechanisms regulating VSMC differentiation.

### (i) OVERVIEW OF THE INVENTION

One aspect of the invention relates to a culture system that promotes differentiation of neural crest cells into myocytic cells, particularly smooth muscle cells. The present culture system can be used to provide a source of, e.g., smooth muscle cells for subsequent use in vitro and in vivo. Moreover, as described herein, the SMC culture can be used to identify genes which are up- or down-regulated as part of the differentiation or migration programs of smooth muscle cells. These identified genes, the products of which are refered to herein as “VSMC proteins” in turn, are useful as diagnositc markers as well as targets for drug development.

Another aspect of the present invention relates to the use of “VSMC therapeutics” of the present invention in treating a broad spectrum of vascular lesions. As used herein, “VSMC therapeutic” refers to a compound which inhibits or potentiates, as the case may be, the biological activity of a VSMC protein. These compounds may be, for example, natural extracts, small organic molecules, nucleic acids, proteins or peptides.

Thus, this invention encompasses a method for inhibiting vascular cellular activity of cells associated with vascular lesion formation in mammals which involves administering an effective dosage of of VSMC therapeutic, e.g., to inhibit proliferation of abnormally dividing vascular smooth muscle cells. Such lesions include, but are not limited to, lesions in the carotid femoral and renal arteries, particularly lesions resulting from renal dialysis fistulas. The methods of the present invention are particularly useful in treating vascular lesions associated with cardiovascular angioplasty. Thus, the VSMC therapeutics can be used in the treatment or prevention of myocardial ischemia, angina, heart failure, atherosclerosis, and as an adjunct in angioplasty for prevention of restenosis and benign prostatic hypertrophy.

In a preferred embodiment, the VSMC therapeutics of the present invention are used for inhibiting or preventing vascular smooth muscle cell proliferation, or restenosis, following vascular intervention or injury, such as angioplasty, vascular bypass surgery, organ transplantation, or other vascular intervention or manipulation. More generally, this invention provides a method for lessening restenosis of body lumens.

### (ii) DEFINITION

For convenience, certain terms employed in the specification, examples, and appended claims are collected here.

The “growth state” of a cell refers to the rate of proliferation of the cell and the state of differentiation of the cell.

“Proliferation,” i.e., of smooth muscle cells, means increase in cell number, i.e., by mitosis of the cells.

“Migration” of smooth muscle cells means movement of these cells in vivo from the medial layers of a vessel into the intima, such as may also be studied in vitro by following the motion of a cell from one location to another (e.g., using time-lapse cinematography or a video recorder and manual counting of smooth muscle cell migration out of a defined area in the tissue culture over time).

The term “modulates”, as in “modulates proliferation or migration of smooth muscle cells” refers to the ability to either inhibit or potentiate.

The phrases “unwanted proliferation” and “abnormal or pathological or inappropriate proliferation” means division, growth or migration of cells occurring more rapidly or to a significantly greater extent than typically occurs in a normally functioning cell of the same type.

As referred to herein, smooth muscle cells and pericytes include those cells derived from the medial layers of vessels and adventitia vessels which proliferate in intimal hyperplastic vascular sites following injury, such as that caused during PTCA.

Characteristics of smooth muscle cells include a histological morphology (under light microscopic examination) of a spindle shape with an oblong nucleus located centrally in the cell with nucleoli present and myofibrils in the sarcoplasm. Under electron microscopic examination, smooth muscle cells have long slender mitochondria in the juxtanuclear sarcoplasm, a few tubular elements of granular endoplasmic reticulum, and numerous clusters of free ribosomes. A small Golgi complex may also be located near one pole of the nucleus. The majority of the sarcoplasm is occupied by thin, parallel myofilaments that may be, for the most part, oriented to the long axis of the muscle cell. These actin containing myofibrils may be arranged in bundles with mitochondria interspersed among them. Scattered through the contractile substance of the cell may also be oval dense areas, with similar dense areas distributed at intervals along the inner aspects of the plasmalemma.

Characteristics of pericytes include a histological morphology (under light microscopic examination) characterized by an irregular cell shape. Pericytes are found within the basement membrane that surrounds vascular endothelial cells and their identity may be confirmed by positive immuno-staining with antibodies specific for alpha smooth muscle actin (e.g., anti-alpha-sml, Biomakor, Rehovot, Israel), HMW-MAA, and pericyte ganglioside antigens such as MAb 3G5 (11); and, negative immuno-staining with antibodies to cytokeratins (i.e., epithelial and fibroblast markers) and von Willdebrand factor (i.e., an endothelial marker).

As used herein, an “agonist” refers to agents which either induce a biological pathway in a cell, e.g., such as by mimicking a ligand for the receptor, as well as agents which potentiate the sensitivity of the cell to the endogenous pathway, e.g., lower the concentrations of ligand required to induce a particular level of receptor-dependent signalling.

A “patient” or “subject” to be treated by the subject method can mean either a human or non-human animal. In preferred embodiments, the subject methods are carried out with human patient.

An “effective amount” of a VSMC therapeutic, with respect to the subject method of treatment, refers to an amount of compound which, when applied as part of a desired dosage regimen brings about a change in the rate of cell proliferation and/or the state of differentiation of a cell so as to produce an amount of muscle cell proliferation or differentiation according to clinically acceptable standards for the disorder to be treated or the cosmetic purpose.

The term “smooth muscle cell markers” refers to gene products which are expressed in smooth muscle, and preferably expressed in smooth muscle cells and not any other cells (at least not neural crest cells).

The term “differential display of mRNA” refers to the generation of a population of mRNA (o cDNA) which is representative of genes whose expression is either up-regulated or down-regulated as compared between two different cells or cell populations.

As used herein, the term “vector” refers to a nucleic acid molecule capable of transporting another nucleic acid to which it has been linked. One type of vector is an genomic integrated vector, or “integrated vector”, which can become integrated into the chromsomal DNA of the host cell. Another type of vector is an episomal vector, i.e., a nucleic acid capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide, including both exon and (optionally) intron sequences. A “recombinant gene” refers to nucleic acid encoding such regulatory polypeptides, which may optionally include intron sequences which are derived from chromosomal DNA. The term “intron” refers to a DNA sequence present in a given gene which is not translated into protein and is generally found between exons. As used herein, the term “transfection” means the introduction of a nucleic acid, e.g., an expression vector, into a recipient cell by nucleic acid-mediated gene transfer.

As used herein, the terms “transduction” and “transfection” are art recognized and mean the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, where anti-sense expression occurs from the transferred gene, the expression of a naturally-occurring form of a protein is disrupted.

### (iii) EXEMPLARY EMBODIMENTS

A. An in vitro System for Rapidly and Uniformly Inducing Immortalized Neural Crest Cells to Smooth Muscle Cell Differentiation.

The components of the in vitro system for vascular smooth muscle cell differentiation from neural crest cells are (1) an immortalized neural crest cell precursor line and (2) smooth muscle cell differentiation medium SMDM. Confirmation of smooth muscle cell differentiation is accomplished by measurements for the presence or absence of markers for smooth muscle cell differentiation: smooth muscle (x-actin, smooth muscle myosin heavy chain, calponin, SM22α, and APEG-1.

Although differentiation and dedifferentation are terms used in the art, the terms will be defined to aid in understanding the invention. By “differentiation” is meant the complex of changes involved in the progressive diversification of the structure and function of a precursor cell into one kind of cell. For a given line of cells, differentiation often results in a continual restriction of the types of transcription that each cell can undertake. By “dedifferentiation” is meant the loss of differentiation, that is, the reversion of specialized cellular structures to a more generalized or primitive condition often as a preliminary to major change. Thus, as explained above, in response to an injury, the vascular smooth muscle cells, both arterial and venous blood vessel cells, which are in a fully differentiated state, will dedifferentiate and begin excessive proliferation, migration, and synthesis. The in vitro system for vascular smooth muscle cell differentiation of this invention is a model that can be used to identify those molecular regulators of smooth muscle cell development and differentiation, and thus, identify compounds that modulate the activity of the molecular regulators.

Immortalized Neural Crest Cell Line

Pluripotent neural crest cells can differentiate into neurons, glia, chondrocytes, melanocytes, and smooth muscle cells. (Stemple and Anderson (1992) Cell 71, 973-985; Shah et al. (1996) Cell 85, 331-343; and Kirby and Waldo (1995) Circ. Res. 77, 211-215.) Various members of the transforming growth factor-B superfamily can instructively promote differentiation of primary cultured neural crest cells into neuronal cells or smooth muscle cells. (Shah, et al., supra). With induction by these factors, however, the neural crest cells do not uniformly differentiate. In other words, some cells will differentiate, but other cells will not differentiate. Without uniform, that is, 100%, or almost 100%, differentiation, it is difficult to analyze markers and molecular regulators. One of the problems solved by this invention is that the neural crest cells differentiate uniformly, that is with 100%, or almost 100%, differentiation.

In the in vitro smooth muscle cell differentiation system of this invention, an immortalized neural crest cell line is employed. In the preferred embodiment, the immortalized neural crest cell line is termed “Monc-1,” an immortalized mouse neural crest cell line that has been retrovirally transfected with the v-myc gene as described in Sommer, et al. (1995) Neuron 15, 1245-1258, and Rao and Anderson (1997) J. Neurobiol. 32, 722-746.

Other immortalized neural crest cell lines, however, may be generated and utilized in the in vitro smooth muscle cell differentiation system of this invention. The selected neural crest cell can be transfected with a variety of other genes that are known to immortalize cells. These genes may include the c-myc and ras-oncogenes. Another preferred method for immortalizing neural crest cells is to obtain neural crest cells from p53 knockout mice. One of skill in the art can determine and use other genes for creating immortalized neural crest cells utilizing known techniques, such as those described in Sambrook, et al., Molecular Cloning: A Laboratory Manual, 2d Ed., Cold Spring Harbor Laboratory Press, Plainview, N.Y. (1989).

The invention will be described below in detail with reference to the immortalized neural crest cell line, Monc-1. However, it is to be understood that other immortalized neural crest cell lines can be used in this invention.

Culture Conditions That Promote Neural Crest Cell to Smooth Muscle Cell Differentiation

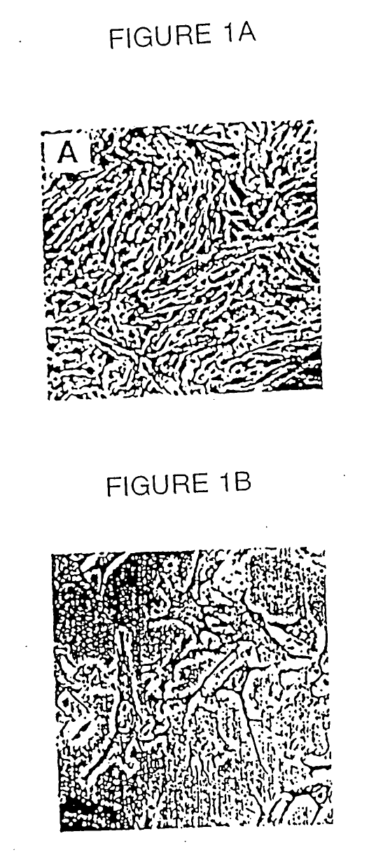

Monc-1 cells on fibronectin-coated plates can be maintained in the undifferentiated state according to the procedures described in Stemple and Anderson, infra, and Sommer, et al., infra. Generally, this medium, termed the “complete medium,” is an L-15 CO₂-based medium supplemented with chick embryo extract. Minor alterations or additions can be made to the complete medium provided that the medium supports the cells in an undifferentiated state. Undifferentiated Monc-1 cells are shown in FIG. 1A.

Confirmation of maintenance of the undifferentiated state of the neural crest cells is determined by the expression of low-affinity nerve growth factor receptor, a marker for undifferentiated neural crest cells. The assay for the low affinity nerve growth factor receptor is described in procedures in Stemple and Anderson, infra, and Sommer, et al., infra. The inventors discovered that changing the culture conditions of the complete medium allow for the differentiation of the neural crest cell down the smooth muscle lineage. This medium is referred to as smooth muscle cell differentiation medium (SMDM). In the preferred embodiment, the SMDM includes the media components listed in Table I supplemented with 10% fetal bovine serum (Hyclone), penicillin (100 units/ml), streptomycin (100 μg/ml), and 25 mM Hepes (pH 7.4). Two different lots of fetal bovine serum were studied. No difference was observed in their ability to induce Monc-1 cell differentiation. The SMDM is not intended to be limited to these components, as equivalents can be utilized and is not intended to be limited to the exact percentages of components, provided that the media effects the Monc-1 phenotype. One skilled in the art would be able to make modifications of the SMDM to support and promote the differentiation of the neural crest cells.

TABLE 1

| Component | mg/L |
|---|---|
| INORGANIC SALTS: | |
| CaCl₂ | 200.00 |
| Fe(NO₂)₃.9H₂O | 0.72 |
| KCl | 400.00 |
| MgSO₄ | 96.00 |
| NaCl | 2200.00 |
| NaHCO₃ | 6800.00 |
| NaH₂PO₄ | 140.00 |
| OTHER COMPONENTS: | |
| Adenine Sulphate | 10.00 |
| Adenosine 5-triphosphate | 1.00 |
| Adenosine 5-phosphate | 0.20 |
| Cholesterol | 0.20 |
| 2-deoxy-D-ribose | 0.50 |
| D-Glucose | 1000.00 |
| Glutathione (reduced) | 0.05 |
| Guanine.HCl | 0.30 |
| Hypoxanthine.Na | 0.40 |
| Phenol Red | 20.00 |
| Ribose | 0.50 |
| Sodium Acetate | 50.00 |
| Thymine | 0.30 |
| TWEEN80 ® | 20.00 |
| Uracil | 0.30 |
| Xanthine.Na | 0.35 |
| AMINO ACIDS: | |
| L-Alanine | 25.00 |
| L-Arginine-HCl | 70.00 |
| L-Aspartic Acid | 30.00 |
| L-Cysteine HCl.H₂O | 0.10 |
| L-Cystine-2HCl. | 26.00 |
| L-Glutamic Acid | 75.00 |
| L-Glutamine | 100.00 |
| Glycine | 50.00 |
| L-Histidine HCl.H₂O | 22.00 |
| L-Hydroxyproline | 10.00 |
| L-Isoleucine | 40.00 |
| L-Leucine | 50.00 |
| L-Lysine.HCl | 70.00 |
| L-Methionine | 15.00 |
| L-Phenylalanine | 25.00 |
| L-Proline | 40.00 |
| L-Serine | 25.00 |
| L-Threonine | 30.00 |
| L-Tryptophan | 10.00 |
| L-Tyrosine.2Na.2H₂O | 58.00 |
| L-Valine | 25.00 |
| VITAMINS: | |
| Ascorbic Acid | 0.05 |
| α-Tacopherol Phosphate | 0.01 |
| (sodium salt) | |
| Biotin | 0.01 |
| Calcifierol | 0.10 |
| D-Ca Pantothenate | 0.01 |
| Choline Chloride | 0.50 |
| Folic Acid | 0.01 |
| I-Inositol | 0.05 |
| Menadione | 0.01 |
| Niacin | 0.025 |
| Niacinamide | 0.025 |
| Para-aminobenzoic Acid | 0.05 |
| Pyridoxal HCl | 0.025 |
| Pyridoxine HCl | 0.025 |
| Riboflavin | 0.01 |
| Thiamine HCl | 0.01 |
| Vitamin A (acetate) | 0.14 |

The SMDM induces a dramatic morphologic change in the Monc-1 neural crest cells. Within 24 hours of incubation in the SMDM, the cells begin to assume a flat, fusiform appearance and the size of the cytoplasm increases. By 4 days of culturing in SMDM, nearly 100% of the cells assume this form as shown in FIG. 1B. Cells cultured in SMDM grow much more slowly than control cells in cultured in complete medium. At confluence, the differentiated cells have the “hill and valley” appearance characteristic of differentiated smooth muscle cell. Exclusive Monc-1 to smooth muscle cell differentiation was indicated by cellular appearance and induction of the smooth muscle cell markers: smooth muscle a-actin, smooth muscle myosin heavy chain, calponin, SM22α, and APEG-1.

Exogenous Activated TGF-β1 Accelerates Smooth Muscle Cell Differentiation

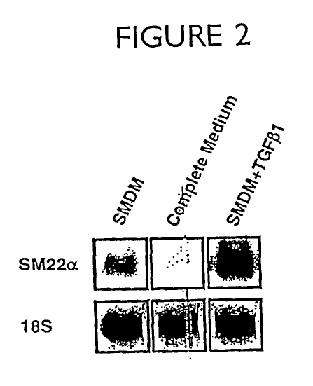

To investigate the TGF-β1 effect on smooth muscle cell differentiation, Monc-1 cells were cultured in SMDM in the presence or absence of TGF-β1 for 33 hours. Total RNA (10 μg) was analyzed by Northern Blotting using a ³²P-labeled SM22α fragment (approximately a 400 base pair mouse SM22α 3′ region). (FIG. 2.) The equal loading of RNA samples was determined by hybridization to a ³²P-labeled oligonucleotide complementary to 18S rRNA. These results show that TGF-β1 accelerated the process of smooth muscle cell differentiation.

Time-Dependent Induction of Smooth Muscle Cell Markers in Response to Neural Crest Cell Differentiation

Confirmation of smooth muscle cell differentiation can be accomplished by determining the presence or absence of known smooth muscle cell markers. Smooth muscle markers include smooth muscle a-actin, smooth muscle myosin heavy chain, calponin, SM22α, and APEG-1. At 4 days of culturing in SMDM, for example, the cell cultures revealed robust expression of the smooth muscle α-actin and calponin genes. The control cells grown in complete medium did not express these markers.

As another control, the markers used for differentiation of the neural crest cells into glial and neuronal cells can be ascertained by determining the presence or absence of immunoreactivity to glial acidic fibrillary protein and peripherin. Smooth muscle cells differentiated from neural crest cells will not stain for glial acidic fibrillary protein or peripherin, although neural crest cells that differentiate into glial and neuronal cells do stain for these proteins.

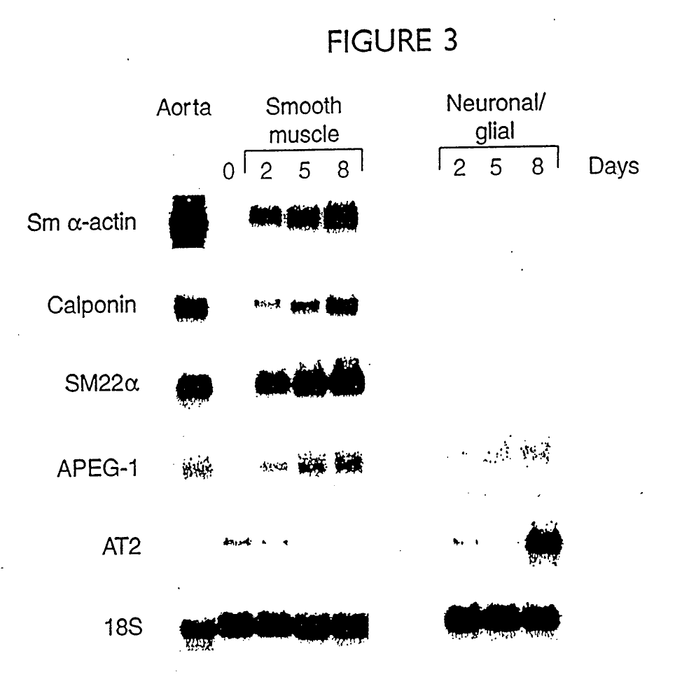

Measuring the expression of the mRNAs for smooth muscle α-actin and calponin, as well as other smooth muscle markers, can be done over the course of Monc-1 cell differentiation in SMDM. FIG. 3 shows the measurement of three smooth muscle markers: smooth muscle α-actin, calponin, SM22α, and APEG-1. In addition, a marker for differentiation to neural cells, angiotensin II receptor (AT2), is also shown. The marker 18S is expressed in both smooth muscle cells and neural cells. The analyses were done at 2, 5, and 8 days after smooth muscle cell differentiation. Messenger RNA from mouse aorta was used as a-positive control for differentiated smooth muscle cell. As a negative control, Monc-1 cells were induced to differentiate down the neuronal and glial pathways in a parallel experiment. Smooth muscle cc-actin and calponin mRNA expression increased as early as 2 days after incubation in SMDM as shown in FIG. 3. Neither mRNA was detected after differentiation down the neuronal pathway.

Another well studied marker of smooth muscle cell is the SM22α gene, which is expressed exclusively in vascular and visceral smooth muscle cell in adult animals. (Kim, et al. (1997) Mol. Cell. Biol. 17, 2266-2278). As with the smooth muscle α-actin and calponin mRNAs, expression of the SM22α mRNA increased after incubation in SMDM as shown in FIG. 3.

APEG-1, a 12.7 kDa nuclear protein preferentially expressed in differentiated vascular smooth muscle cell, was cloned recently as described in Hsieh, et al., (1996) J. Biol. Chem. 271, 17354-17359. Its expression is high in arterial smooth muscle cell in vivo but quickly and completely becomes downregulated in dedifferentiated arterial smooth muscle cell both in vitro and in vivo. Expression of APEG-1 mRNA also increased during Monc-1 cell culture in SMDM as shown in FIG. 3. APEG-1 thus represents a sensitive marker for differentiated vascular smooth muscle cell. The expression of APEG-1 in Monc-1 cells differentiated in SMDM indicates that these cells share properties with differentiated vascular smooth muscle cells.

The marker AT2 is expressed in the vasculature and many other tissues during embryogenesis (Lenkei, et al. (1996) J. Comp. Neurol. 373, 322-339; Shanmugam, et al. (1995) Kidney Int. 47, 1095-1100; and, Shanmugam and Sandberg, (1996) Cell Biol. Int. 20, 169-176.) Although AT2 expression is downregulated in the adult vasculature, it continues in adult neuronal cells. As FIG. 3 shows, AT2 mRNA was expressed in undifferentiated Monc-1 cells (day 0). Although AT2 mRNA was still visible after 2 days of incubation in SMDM, it disappeared after 5 and 8 days of incubation. AT2 mRNA expression increased as neuronal cell and glial cell differentiation progressed.

B. Identification of Regulators of Smooth Muscle Cell Differentiation.

The gene products that regulate smooth muscle cell differentiation are termed “nodal regulators” and are typically proteins or polypeptides, e.g., “VSMC proteins”. Nodal regulators are involved in a number of smooth muscle cell differentiation activities. Regulation of cell differentiation activity encompasses transcription, RNA processing, translation, as well as events associated with protein expression, such as expression of secreted substances, and control of metabolic activity, such as cell multiplication, mitosis, replication, and apoptosis. The in vitro smooth muscle cell differentiation system was used to identify different nodal regulators, as well as other molecular regulators. First, the induction of smooth muscle myosin heavy chain expression in response to Monc-1 cell differentiation was studied. It was also determined whether SM22α promoter activity was induced in smooth muscle cell differentiation.

Induction of Smooth Muscle Myosin Heavy Chain Expression in Response to Monc-1 Cell Differentiation

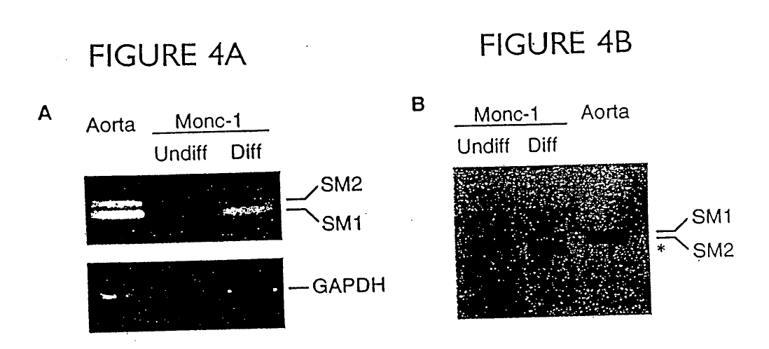

To determine whether smooth muscle myosin heavy chain, a specific marker of differentiated SMC, was expressed in Monc-1 cells, reverse transcription PCR was used. Specifically, a pair of primers that amplify 324 base pair and 363 base pair fragments of the SM1 and SM2 isoforms, respectively. The SM1 and SM2 DNA fragments were both amplified from reverse transcribed mouse aorta RNA (FIG. 4A.) SM1 was amplified from differentiated but not undifferentiated Monc-1 cell RNA. Primers for glyceraldehyde-3-phosphate dehydrogenase (GAPDH) were used to amplify a specific band from all RNA samples. SM1 and SM2 were both detected in samples prepared from mouse aortas by high resolution Western analysis with an antibody to smooth muscle myosin heavy chain (Groschel-Stewart, et al. (1976) Histochemistry 46, 229-236)(FIG. 4B). Although undifferentiated Monc-1 cells expressed only non-muscle myosin heavy chain (FIG. 4B, asterisk), differentiated Monc-1 cells expressed SM1. Taken together, these data indicate the presence of SM1 in SMC differentiated from Monc-1 cells.

Induction of SM22α Promoter Activity by Neural Crest Cell to Smooth Muscle Cell Differentiation

To gain further insight into the molecular mechanisms regulating Monc-1 cell differentiation, the effect of the SM22a promoter on Monc-1 differentiation was studied. The SM22α promoter was chosen because the molecular mechanisms regulating expression of the SM22α gene are very well characterized. A 0.4 kb region of the SM22α promoter, within which lie two CArG elements, has been shown to mediate vascular smooth muscle cell-specific expression in transgenic mice. This cis-acting element, the CArG box (CC A/T₆GG), is critical for expression of SM22α in vascular smooth muscle cells in vitro and in vivo. Because mutation of the proximal CArG element eliminates all SM22α expression in transgenic animals, this element appears to be necessary and sufficient for directing high-level expression restricted to the smooth muscle cell lineage. (Kim, et al. (1997) Mol. Cell. Biol. 17, 2266-2278; Li, et al., (1996) J. Cell Biol. 132, 849-859; and Moessler, et al. (1996) Development 122, 2415-2425).

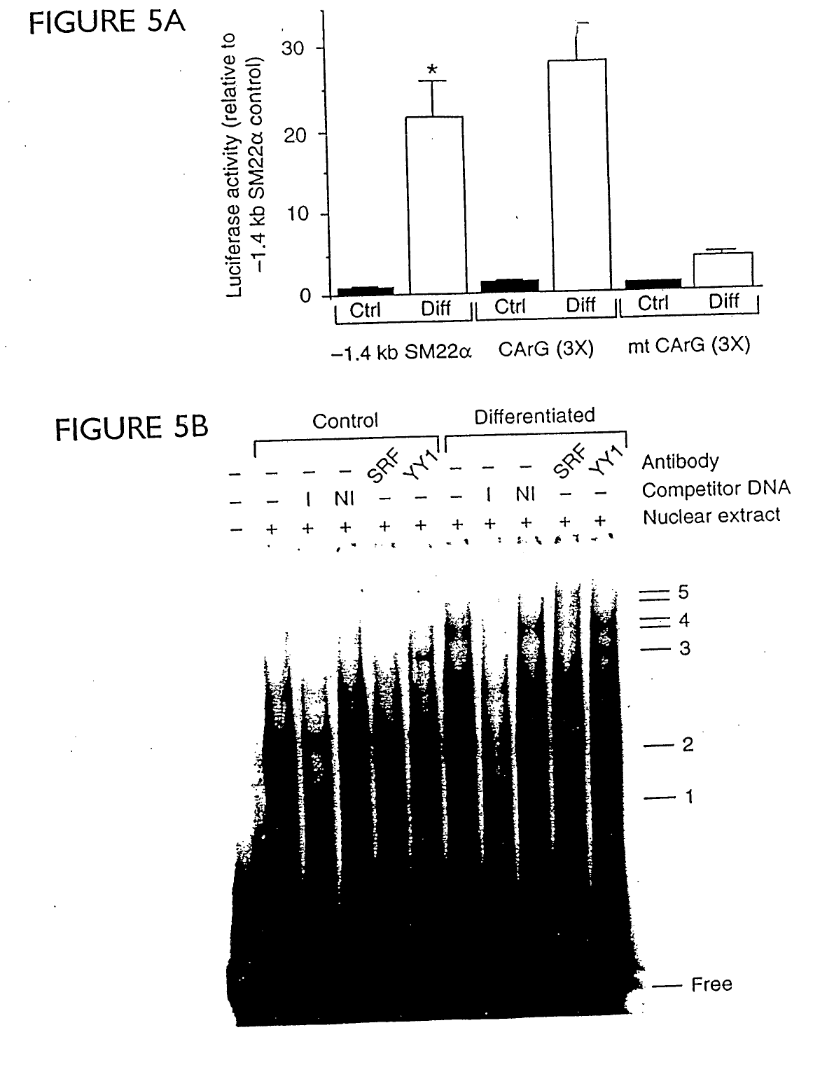

To determine whether the same cis-acting element is critical to induction of SM22α in Monc-1 cells differentiated down the smooth muscle lineage, transient transfection assays were performed with the SM22α promoter. First, a luciferase reporter construct, −1.4 kb SM22α, was generated containing 1.4 kb of the SM22α promoter. Next, Monc-1 cells were transfected with the SM22α construct by electroporation and cultured in complete medium or SMDM. Luciferase activity was measured in cell lysates 72 hours after transfection. The −1.4 kb SM22α promoter was minimally active in undifferentiated Monc-1 cells as shown in FIG. 5A. After differentiation of the Monc-1 cells into smooth muscle cells, however, SM22α promoter activity markedly increased by 20-30-fold.

Two other reporter constructs were generated to see whether the CArG element was critical to regulation of SM22α promoter activity after Monc-1 cell to smooth muscle cell differentiation in vitro. The first, CArG (3X), contained three SM22α CArG elements upstream of the luciferase reporter gene, and the second, mtCArG (3X), contained three SM22α CArG elements in which the core sequence had been modified from 5′CCAAATATGG3′ to 5′CCACACATGG3′. This mutated sequence cannot function as an enhancer in smooth muscle cell. Like the activity of the native SM22α promoter, the activity of the multimerized CArG reporter construct increased dramatically as shown in FIG. 5A after Monc-1 cell to smooth muscle cell differentiation, again by at least 20-fold. Mutation of the CArG box markedly decreased differentiation-induced activation of the SM22α promoter after Monc-1 cell to smooth muscle cell differentiation. Thus, this 20-30 fold increase in the activity of the smooth muscle cell-specific promoter SM22α coincided with smooth muscle cell differentiation.

Induction of Serum Responsive Factor DNA-Binding Activity by Neural Crest Cell to Smooth Muscle Cell Differentiation

The in vitro differentiation system was used to determine whether specific trans-acting factors may be induced after Monc-1 differentiation that would bind to the CArG element and thereby regulate SM22α expression. Gel mobility shift assays were conducted by using the CArG (3X) oligonucleotide as a probe with nuclear extracts from differentiated vascular smooth muscle cells and Monc-1 cells. Five specific DNA-protein complexes as shown in FIG. 4B were revealed, three of which are natural (complexes 1, 2, and 4) and two of which are supershifted (complexes 3 and 5). Complexes 1 and 4 appeared in nuclear extracts from differentiated Monc-1 cells but not in those from undifferentiated cells. Complex 2 was present under both conditions but appeared to intensify after differentiation. Antibody supershift experiments showed that complex 4 (shifted to 5) contains a protein antigenically identical or related to senun responsive factor, whereas complex 2 (shifted to 3) contains a protein related to YY1. Complex 1 was the only complex visible solely in nuclear extracts from Monc-1 cells after differentiation. The protein contained in this novel complex is a molecular regulator of this invention.

Thus, by gel mobility shift analysis, new DNA-protein complexes in nuclear extracts prepared from differentiated Monc-1 cells were identified. One of the new complexes contained serum responsive factor (SRF) and had CArG element-binding activity.

These results show that the activity of the SM22α promoter and a multimerized reporter construct containing the proximal CArG element, but not that of a construct containing a mutated CArG element, increased markedly in Monc-1 cells after differentiation down the smooth muscle lineage as seen in FIG. 5A. These data suggest that specific factors induced by differentiation bind to the CARG element and activate the SM22α promoter. Indeed, three DNA-protein complexes as shown in FIG. 5B were seen in nuclear extract from differentiated but not undifferentiated Monc-1 cells by gel mobility shift analysis, and the intensity of a fourth complex increased in extract from differentiated Monc-1 cells. These four complexes contain proteins identical or antigenically related to SRF and YY1, two factors known to synergistically activate the CArG box of SM22α and other genes. Together, these studies of the SM22α promoter suggest that the genetic program normally instituted in smooth muscle cells in vivo is recapitulated during Monc-1 cell to smooth muscle cell differentiation in vitro.

Isolation of Genes Coding for Molecular Regulators of Vascular Smooth Muscle Cell Differentiation

Thus, the novel in vitro vascular smooth muscle cell differentiation system may be used as a method to identify other regulators. More particularly, the disclosed differentiation system may be used to systematically isolate and purify and clone genes important for regulating vascular smooth muscle cell differentiation. For example, the transcription factors present in complex 1 previously identified by gel mobility shift assay can be purified and cloned. In addition, two powerful molecular cloning methods can be employed, to systematically isolate the regulators: differential display and genetic screening techniques.

For example, such differential display techniques as described in U.S. Pat. Nos. 5,827,658 and 5,814,445 and Liang et al., (1993) Nucleic Acids Res. 21: 3269-75; Liang and Pardee (1992) Science 257: 967-971; and Chapman et al. (1995) Mol. Cell Endocrinol., 108:R1-R7.

Likewise, suppreosion PCR can be used. Briefly, cDNA adaptors are engineered to prevent undesirable amplification during PCR (1, 2). Suppression occurs when complementary sequences are present on each end of a single-stranded cDNA. During each primer annealing step, the hybridization kinetics strongly favor (over annealing of the shorter primers) the formation of a pan-like secondary structure that prevents primer annealing. When occasionally a primer anneals and is extended, the newly synthesized strand will also have the inverted terminal repeats and form another pan-like structure. Thus, during PCR, nonspecific amplification is efficiently suppressed, and specific amplification can proceed normally. Lukyanov et al. (1994) Biorganic Chem. (Russian) 20:701-704; and Siebert et al. (1995) Nucleic Acids Res. 23:1087-1088. Commercially available kits include CLONTECH's Marathon™ cDNA Amplification (#K1802-1) and GenomeWalker™ Kits.

Moreover, the importance of candidate nodal regulators in vascular remodeling can be tested by other genetic approaches, including Northern analysis with mRNA isolated from injured carotid arteries and the like.

Differential Display by the Hieroglyph System

RNA isolated from Monc-1 cells before and after (3, 6, 12, 24, and 72 hours) differentiation into smooth muscle cells. This RNA is used as templates for differential display with the HIEROGLYPH™ mRNA Profile Kit (Genomyx, Foster City, Calif.) or the recently developed CHIP technology. Differential display is a technique that allows a comparison of an RNA of two different cell types, i.e., undifferentiated neural crest cells and differentiated smooth muscle cells, at different time points, so as to obtain a fingerprint of these cells' mRNA over time. RNA that has been transcribed from genes that appear or disappear during Monc-1 cell differentiation is a preferred embodiment for differential display. Genes that appear (putative molecular regulator for differentiation) or disappear (putative anti-differentiation gene) in differentiated Monc-1 cells are of particular interest.

Full length DNA clones from these mRNA from differential display gels were isolated by RACE or library screening. Because the Hieroglyph differential display system generates longer cDNA fragments, one of ordinary skill may determine the partial open reading frames of the cloned genes. Both the nucleotide sequence and the partial amino acid sequences are used to search the GenBank and TIGR databases. Priority for full-length DNA isolation is made for genes preferentially expressed in vascular smooth muscle cells as well as genes with homology to transcription factors, signaling molecules, or kinases/phosphatases that may be regulators of smooth muscle cell differentiation.

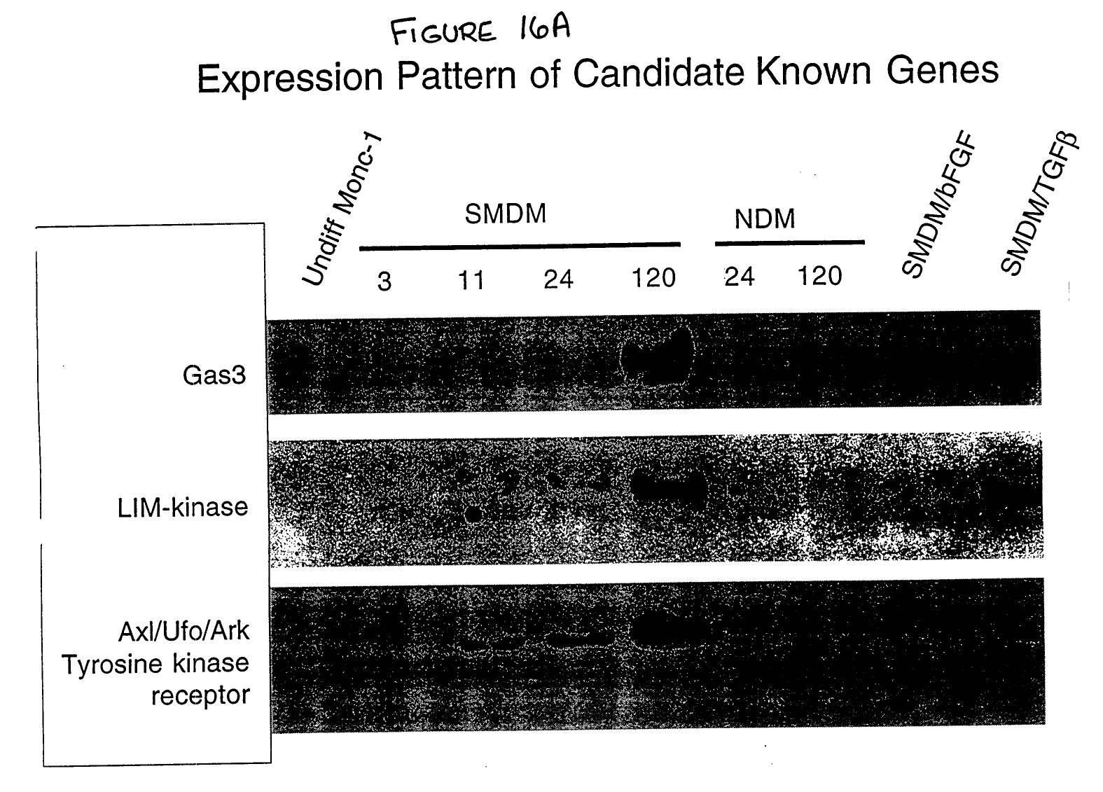

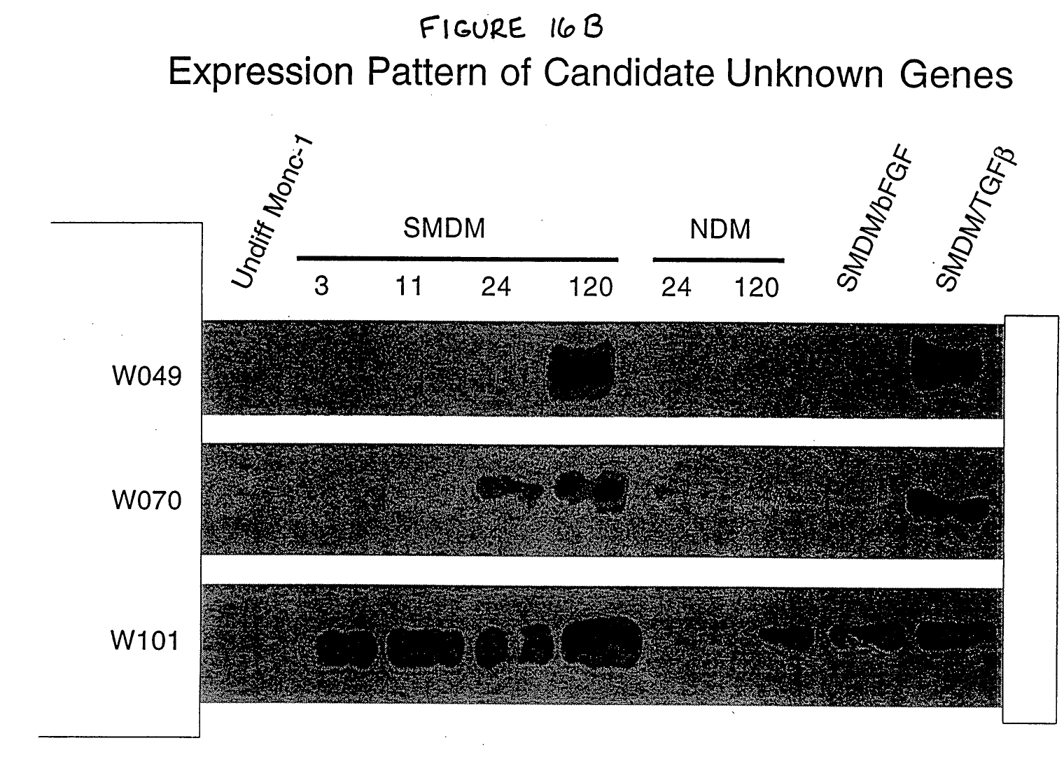

For example, several bands with upregulated or downregulated expression patterns during smooth muscle cell differentiation were identified and isolated. Briefly, these bands were cut out from a differential display gel and re-amplified by PCR. These PCR products were analyzed on 1% agarose gels and purified by a QIAEX II Agarose Gel Extraction kit (Qiagen, Chatsworth, Calif.). These fragments were then sequences by Thermo Sequence radiolabeled terminator cycle sequencing kit (Amersham, Cleveland, Ohio) and homology search with GenBank and TIGR databases was done via the internet. By sequence analysis, many of these clones were identified as sharing homology with known genes. Other clones have yet to be identified. Accordingly, smooth muscle cell differentiating regulating genes identified by the differential display method described herein may include (1) smooth muscle markers: smooth muscle γ-actin, RCL-A myosin regulatory chain; (2) growth arrest related genes: Ufo/Ax1/Ark, Gas3, ccal, 204/202 Interferon inducible protein; (3) TGF-β related genes: LTBP-1, TSC-36, Decorin; (4) transcriptional factor/nucleic proteins: c-jun, FRA2, prothymosin-α; (5) kinase: Integrin binding protein kinase, LIM-kinase 2b; (6) others: Osteonectin/SPARC, rrg/lysyl oxidase. FIGS. 16A and 16B shows the expression pattern for certain of these genes.

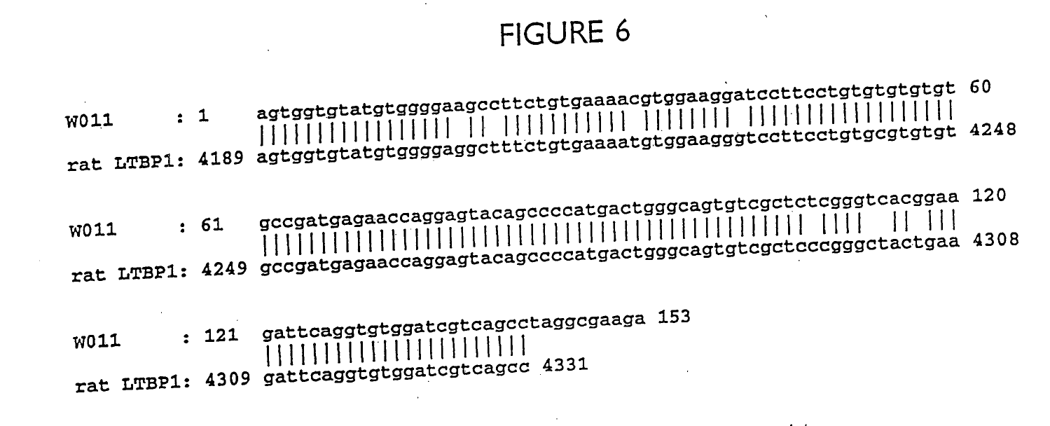

Among the cloned genes, one fragment, termed W011, was amplified by a combination of anchored primer 4 (5′ACGACTCACTATAGGGCTTTTTTTTTTGT-3′) and Arbitrary primer 8(5′-ACAATTTCACACAGGATGGTAAACCC-3′) and estimated as 760 base pairs long. The W011 fragment was sequenced 153 bp of the 5′ end and was found to share significant homology with rat latent TGF-β binding protein 1 (LTBP-1) (GenBank accession number: M55431, 6244 base pairs, homology 94.4%) and human LTBP-1. A comparison of the nucleic acid sequences of W011 with rat LTBP-1 is shown in FIG. 6. Considering this homology data, it is highly possible that W011 is a mouse homologue of LTBP-1, a latent TGF-β1 binding protein.

LTBP-1 is one of three distinct components of the latent TGF-β1 complex. Latent TGF-β1 is the high molecular weight, biologically inactive form that is usually secreted by normal cells. This complex contains (1) the mature TGF-β1 that is biologically active; (2) the TGF-β1 latent associated peptide (LAP) that is sufficient for TGF-β1 latency; and (3) the LTBP-1. LTBP-1 is the best characterized of these proteins (Kanzaki, et al., (1990) Cell 61, 1051-1061; Tsuji, et al. (1990) Proc. Natl. Acad. Sci. 87, 8835-8839.). It is a glycoprotein that associates with LAP by disulfide bonds. LTBP-1 is assumed to play a strategic role in the assembly, secretion, and activation of latent TGF-β1. Although the activity of TGF-β1 is important in wound healing, increasing evidence links excessive TGF-β1 activity to a wide variety of fibrotic diseases, such as glomerulonephritis, arteriosclerosis, and liver cirrhosis (Tamaki, et al., (1995) Lab. Invest. 73, 81-89; Waltenberger, et al., (1993) Am. J. Pathol. 142, 71-78; Border and Ruoslahi, (1992) J. Clin. Invest. 90, 1-7). Although it has been shown that LTBP-1 may facilitate activation of latent TGF-β1 in smooth muscle cells (Flaumenhaft, et al., (1993) J. Cell Biol. 120, 995-1002.), its role in smooth muscle cell differentiation as a regulator of TGF-β1 has not been investigated.

Characterization of the genes that are identified by differential display (or previously by gel mobility shift assay) are further studied in a variety of ways. First, the expression pattern and size of an mRN4A is determined by Northern hybridization. The expression pattern of the putative molecular regulators is confirmed in cultured undifferentiated and differentiated Monc-1 cells in culture, normal and atherosclerotic arterial tissue in both mice and humans.

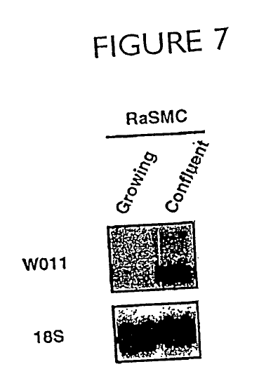

For example, in order to examine the expression pattern of LTBP-1 in differentiated smooth muscle cells, Northern blot analysis was performed. Briefly, RNA was isolated from growing rat arterial smooth muscle cells in primary culture and compared to RNA isolated from confluent rat arterial smooth muscle cells. The RNA was analyzed by Northern blotting, using the W011 probe. (FIG. 7.) Two major signals were observed and found to be increased approximately twelve-fold with confluency of the rat arterial smooth muscle cells. The equal loading of RNA samples was determined by hybridization to a ³²P-labeled oligonucleotide complementary to 18S rRNA. The signal intensity was quantified using a Phosphoimager and ImageQuant software (Molecular Dynamics, Sunnyvale, Calif.). These results suggest that LTBP may play a role in smooth muscle cell differentiation by regulating TGF-β1.

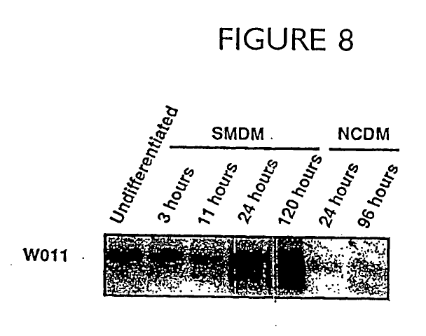

Further, in order to confirm the induction of the LTBP-1 expression pattern during smooth muscle cell differentiation, another Northern blot analysis was performed. For this experiment, RNA was isolated from undifferentiated Monc-1 cells, and Monc-1 cells cultured in SMDM for 3, 11, 24, and 120 hours, as well as from Monc-1 cells cultured in neuronal differentiation medium for 24 and 96 hours. Total RNA (10 μg) was transferred to a membrane, and hybridized with a ³²P-labeled W011 fragment. (FIG. 8.) Two major bands were observed that increase in abundance with smooth muscle cell differentiation, but not with neuronal cell differentiation.

Second, the biology of these new cDNAs in cultured rat aortic smooth muscle cells and Monc-1 calls is studied by generating clones expressing the candidate genes in a constitutive or an inducible manner. Alternatively, microinjection of the plasmids encoding these genes is a fast screening technique in cases where many genes are isolated. The constitutive/inducible clones or microinjected cells harboring these genes are studied for their ability to proliferate, migrate, and synthesize extracellular matrix in comparison with control clones. The-differentiation-promoting genes inhibit proliferation, migration, or synthesis of extracellular matrix.

For selected genes, such as potential growth inhibitors, an antisense or dominant-negative approach is taken. Antisense oligonucleotides to these genes may be made to block, for example, the proliferation, migration, or synthesis of the extracellular matrix. Also, the biological effects of the candidate genes may be judged by their ability to enhance or inhibit differentiation down the vascular smooth muscle cell pathway. The biological effects of these genes is also tested on Monc-1 cell differentiation, although a retroviral approach is taken because it is more difficult to transfect Monc-1 cells.

Third, the biology of these molecular regulator genes is studied in vivo. Genes that potentially inhibit or enhance differentiation in vascular smooth muscle cells or Monc-1 cells in vitro are further studied in vivo. To conduct these studies, the cloned genes are overexpressed by using an adenovirus or a transgenic approach with a vascular smooth muscle cell-specific promoter. For example, overexpression of a differentiation growth inhibitory gene (or the antisense version of a differentiation/growth inhibitory gene) should prevent proliferation of vascular smooth muscle cell and formation of neointima in response to vascular injury. The overexpression of, or antisense blocking of, such genes may be suitable as gene therapy methods. The function of these candidate molecular regulatory genes is studied by gene deletion or different mouse model systems.

Genetic Screening for Vascular Smooth Muscle Cell Differentiation or Dedifferentation Genes

This method uses a genetic screen to isolate genes that will confer a differentiated phenotype. This strategy involves the SM22α 5′-flanking. SM22α is active only in differentiated rat aortic smooth muscle cells or Monc-1 cells, but is inactive in dedifferentiated rat aortic smooth muscle cells or undifferentiated Monc-1 cells. Consequently, these latter cell types do not express a hygromycin resistance gene driven by SM22α 5′-flanking sequence in a transfected plasmid. Introduction, therefore, of genes by transfection of a retroviral cDNA library that confer a differentiation phenotype in these cells will activate the SM22α 5′-flanking sequence and allow expression of the hygromycin resistance gene. These cells are selected by hygromycin and the transfected cDNA encoding the differentiation or dedifferentation molecular regulator gene is then harvested by PCR.

To conduct this genetic screening method, a plasmid containing the SM22α promoter sequences 5′ to the hygromycin resistance gene, designated pSM22α-Hygro is generated. This plasmid also contains a zeocin selective marker driven by the CMV promoter. A cDNA library from mRNA prepared from normal rat or human aorta containing primarily differentiated vascular smooth muscle cells is generated with the retroviral vector pLNCX.

To screen for vascular smooth muscle cell differentiation genes, the following procedures may be used.
1. Rat aortic smooth muscle cells or Monc-1 cells are transfected with (at passage 2) pSM22α-Hygro by electroporation. Integration of the pSM22α-Hygro is determined by the cells′ resistance to zeocin, and integration of the plasmid DNA is confirmed by Southern hybridization analysis. Only clones that do not express hygromycin at all or at very low levels are used for the subsequent studies below.
2. The clones are transduced with libraries in retroviruses. Clones that are successfully transduced are selected with G-418.
3. The transduced rat aortic smooth muscle cell clones are selected with hygromycin. Selection is followed by recovery of the transduced cDNA by reverse transcription PCR with specific primers.
4. The full length cDNA clones are obtained and characterized as described in the section of differential display.
   C. Identification of Compounds that Inhibit or Regulate the Molecular Regulators Identified from the in vitro Smooth Muscle Cell Differentiation System.

Another aspect of the invention is directed to the identification of agents capable of modulating the growth state of smooth muscle cell, e.g., of differentiation and proliferation. These agents include, but are not limited to, compounds that either potentiate or inhibit an intrinsic enzymatic activity of a VSMC protein or a complex including a VSMC protein, compounds that interfere with the interaction of the VSMC protein with other protein(s) or nucleic acid, and compounds comprising forms of the VSMC proteins that are altered (mutated) to provide dominant loss-of-function or gain-of-function activity.

In this regard, the present invention provides assays for identifying agents which are either agonists or antagonists of the normal cellular function of the subject VSMC proteins, or of the role of those proteins in the pathogenesis of normal or abnormal cellular proliferation and/or differentiation of smooth muscle cells and disorders related thereto. In one embodiment, the assay evaluates the ability of a compound to modulate binding of a VSMC protein with other proteins, DNA or RNA. In other embodiments, the subject assay detects compounds which modulate an enzymatic activity of a VSMC protein. Compounds identified by the present assay can be used, for example, in the treatment of proliferative and/or differentiative disorders, and to modulate apoptosis.

Agents to be tested for their ability to act as agonists or antagonists of a VSMC protein can be produced, for example, by bacteria, yeast or other organisms (e.g. natural products), produced chemically (e.g. small molecules, including peptidomimetics), or produced recombinantly. In a preferred embodiment, the test agent is a small organic molecule having a molecular weight of less than about 2,000 daltons. A high speed screen for agents that bind directly to the molecular regulator may employ immobilized or “tagged” combinatorial libraries (or libraries which otherwise readily deconvoluted).

Agents that are identified as active in the drug screening assay are candidates to be tested for their capacity to block smooth muscle cell differentiation activity. As described below, these agents would be useful for treating or preventing stenosis, arteriosclerosis or other disorder involving aberrant growth of smooth muscle cells by inhibiting or regulating the activity of smooth muscle cell differentiation.

A variety of assay formats will suffice and, in light of the present disclosure, those not expressly described herein will nevertheless be comprehended by one of ordinary skill in the art. For instance, the assay can be generated in many different formats, and include assays based on cell-free systems, e.g. purified proteins or cell lysates, as well as cell- based assays which utilize intact cells. Simple binding assays can also be used to detect agents which, such as those which detect compounds able to potentiate or disrupt protein- protein or protein-DNA interaction involving a VSMC protein.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

Accordingly, in an exemplary screening assay of the present invention, a reaction mixture is generated to include a VSMC protein, test compound(s), and a “target molecule”, e.g., a protein or nucleic acid which interacts with the VSMC protein or which is a substrate of an enzymatic activity of the VSMC protein. Detection and quantification of interaction, or substrate conversion (as appropriate) of the VSMC protein with the target molecule provides a means for determining a compound's efficacy at inhibiting or potentiating interaction between the VSMC protein and the target molecule. The efficacy of the compound can be assessed by generating dose response curves from data obtained using various concentrations of the test compound. Moreover, a control assay can also be performed to provide a baseline for comparison. In the control assay, interaction of the VSMC protein and target molecule is quantitated in the absence of the test compound.

Interaction between the VSMC protein and the target molecule may be detected by a variety of techniques. Modulation of the formation of complexes can be quantitated using, for example, detectably labeled proteins such as radiolabeled, fluorescently labeled, or enzymatically labeled VSMC proteins, by immunoassay, by chromatographic detection, or by detecting the intrinsic activity of the acetylase.

Typically, it will be desirable to immobilize either the VSMC protein or the target molecule to facilitate separation of complexes from uncomplexed forms of one or both of the proteins, as well as to accommodate automation of the assay. Binding of VSMC protein to the target molecule, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/VSMC protein (GST/VSMC protein) fusion proteins can be adsorbed onto glutathione sepharose beads (Sigma Chemical, St. Louis, Mo.) or glutathione derivatized microtitre plates, which are then combined with the cell lysates, e.g. an ³⁵S-labeled, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes can be dissociated from the matrix, separated by SDS-PAGE, and the level of target molecule found in the bead fraction quantitated from the gel using standard electrophoretic techniques.

Other techniques for immobilizing proteins and other molecules on matrices are also available for use in the subject assay. For instance, either the VSMC protein or target molecule can be immobilized utilizing conjugation of biotin and streptavidin. For instance, biotinylated VSMC protein molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the VSMC protein, but which do not interfere with the interaction between the VSMC protein and target molecule, can be derivatized to the wells of the plate, and the VSMC protein trapped in the wells by antibody conjugation. As above, preparations of an target molecule and a test compound are incubated in the VSMC protein-presenting wells of the plate, and the amount of complex trapped in the well can be quantitated. Exemplary methods for detecting such complexes, in addition to those described above for the GST-immobilized complexes, include immunodetection of complexes using antibodies reactive with the target molecule, or which are reactive with the VSMC protein and compete with the target molecule; as well as enzyme-linked assays which rely on detecting an enzymatic activity associated with the VSMC protein or target molecule, either intrinsic or extrinsic activity. In the instance of the latter, the enzyme can be chemically conjugated or provided as a fusion protein with the VSMC protein or target molecule. To illustrate, the target molecule can be chemically cross-linked or genetically fused (if it is a polypeptide) with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating proteins trapped in the complex, antibodies against the protein, such as anti-VSMC protein antibodies, can be used. Alternatively, the protein to be detected in the complex can be “epitope tagged” in the form of a fusion protein which includes, in addition to the VSMC protein sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

An exemplary drug screening assay of the present invention includes the steps of (a) forming a reaction mixture including: (i) a target molecule, such as a latency associated peptide (LAP) or TGFβ, (ii) a VSMC protein, such as a latent TGFβ binding protein (LTBP-1) and (iii) a test compound; and (b) detecting interaction of the target molecule and VSMC protein. A statistically significant change (potentiation or inhibition) in the interaction of the VSMC proteins in the presence of the test compound, relative to the interaction in the absence of the test compound, indicates a potential agonist (mimetic or potentiator) or antagonist (inhibitor) for the test compound. The reaction mixture can be a cell-free protein preparation, e.g., a reconsistuted protein mixture or a cell lysate, or it can be a recombinant cell including a heterologous nucleic acid recombinantly expressing the VSMC protein.

Where the VSMC protein is a receptor, or participates as part of an oligomeric receptor complex, e.g., which complex includes other protein subunits, the cell-free system can be, e.g., a cell membrane preparation, a reconstituted protein mixture, or a liposome reconstituting the receptor. For instance, the protein subunits of a receptor complex including the VSMC protein can be purified from detergent extracts from both authentic and recombinant origins can be reconstituted in in artificial lipid vesicles (e.g. phosphatidylcholine liposomes) or in cell membrane-derived vesicles (see, for example, Bear et al. (1992) Cell 68:809-818; Newton et al. (1983) Biochemistry 22:6110-6117; and Reber et al. (1987) J Biol Chem 262:11369-11374). The lamellar structure and size of the resulting liposomes can be characterized using electron microscopy. External orientation of the receptor in the reconstituted membranes can be demonstrated, for example, by immunoelectron microscopy. The interaction of a ligand or test compound with liposomes containing such VSMC protein complexes and liposomes without the protein can be compared in order to identify potential modulators of the receptor.

In yet another embodiment, the drug screening assay is derived to include a whole cell expressing a VSMC protein. The ability of a test agent to alter the activity of the VSMC protein can be detected by analysis of the recombinant cell. For example, agonists and antagonists of the VSMC protein biological activity can by detected by scoring for alterations in growth or differentiation (phenotype) of the cell. General techniques for detecting each are well known, and will vary with respect to the source of the particular reagent cell utilized in any given assay. For the cell-based assays, the recombinant cell is preferably a metazoan cell, e.g., a mammalian cell, e.g., an insect cell, e.g., a xenopus cell, e.g., an oocyte. In preferred embodiment, the cell is a mammalian cell of myocytic phenotype or origin. In other embodiments, where the VSMC protein is a receptor, the receptor can be reconsituted in a yeast cell.

In addition to morphological studies, change(s) in the level of an intracellular second messenger responsive to activities dependent on the VSMC protein can be detected. For example, in various embodiments the assay may assess the ability of test agent to cause changes in phophorylation patterns, adenylate cyclase activity (cAMP production), GTP hydrolysis, calcium mobilization, and/or phospholipid hydrolysis (IP3, DAG production). By detecting changes in intracellular signals, such as alterations in second messengers or gene expression, candidate agonists and antagonists to VSMC protein-dependent signaling can be identified.

VSMC proteins may regulate the activity of phospholipases. Inositol lipids can be extracted and analyzed using standard lipid extraction techniques. Water soluble derivatives of all three inositol lipids (IP₁, IP₂, IP₃) can also be quantitated using radiolabelling techniques or HPLC.

The mobilization of intracellular calcium or the influx of calcium from outside the cell may be dependent on a VSMC protein. Calcium flux in the reagent cell can be measured using standard techniques. The choice of the appropriate calcium indicator, fluorescent, bioluminescent, metallochromic, or Ca⁺⁺-sensitive microelectrodes depends on the cell type and the magnitude and time constant of the event under study (Borle (1990) Environ Health Perspect 84:45-56). As an exemplary method of Ca⁺⁺detection, cells could be loaded with the Ca⁺⁺ sensitive fluorescent dye fura-2 or indo-1, using standard methods, and any change in Ca⁺⁺ measured using a fluorometer.

In certain embodiments of the assay, it may be desirable to screen for changes in cellular phosphorylation. The ability of compounds to modulate serine/threonine kinase or tyrosine kinase activation could be screened using colony immunoblotting (Lyons and Nelson (1984) PNAS 81:7426-7430) using antibodies against phosphorylated serine, threonine or tyrosine residues. Reagents for performing such assays are commercially available, for example, phosphoserine and phosphothreonine specific antibodies which measure increases in phosphorylation of those residues can be purchased from comercial sources.

Certain of the VSMC protein may set in motion a cascade involving the activation and inhibition of downstream effectors, the ultimate consequence of which is, in some instances, a detectable change in the transcription or translation of a gene. By selecting transcriptional regulatory sequences from such target genes, e.g., that are responsible for the up- or down-regulation of these genes, and operatively linking such promoters to a reporter gene, the present invention provides a transcription based assay which is sensitive to the ability of a specific test compound to influence signalling pathways dependent on the VSMC protein.

In an exemplary embodiment, the subject assay comprises detecting, in a cell-based assay, change(s) in the level of expression of a gene controlled by a transcriptional regulatory sequence responsive to signaling by a VSMC protein. Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on signaling by the VSMC protein. Expression of the reporter gene, thus, provides a valuable screening tool for the development of compounds that act as agonists or antagonists of VSMC protein-dependent signalling.

In practicing one embodiment of the assay, a reporter gene construct is inserted into the reagent cell in order to generate a detection signal dependent on second messengers generated by the VSMC protein. Typically, the reporter gene construct will include a reporter gene in operative linkage with one or more transcriptional regulatory elements responsive to signal transduction from the VSMC protein, with the level of expression of the reporter gene providing the detection signal. The amount of transcription from the reporter gene may be measured using any method known to those of skill in the art to be suitable. For example, mRNA expression from the reporter gene may be detected using RNAse protection or RNA-based PCR, or the protein product of the reporter gene may be identified by a characteristic stain or an intrinsic activity. The amount of expression from the reporter gene is then compared to the amount of expression in either the same cell in the absence of the test compound or it may be compared with the amount of transcription in a substantially identical cell that lacks the target receptor protein. Any statistically or otherwise significant difference in the amount of transcription indicates that the test compound has in some manner altered the inductive activity of the VSMC protein.

As described in further detail below, in preferred embodiments the gene product of the reporter is detected by an intrinsic activity associated with that product. For instance, the reporter gene may encode a gene product that, by enzymatic activity, gives rise to a detection signal based on color, fluorescence, or luminescence. In other preferred embodiments, the reporter or marker gene provides a selective growth advantage, e.g., the reporter gene may enhance cell viability, relieve a cell nutritional requirement, and/or provide resistance to a drug. Many reporter genes are known to those of skill in the art and others may be identified or synthesized by methods known to those of skill in the art. A reporter gene includes any gene that expresses a detectable gene product, which may be RNA or protein.

Preferred reporter genes are those that are readily detectable. The reporter gene may also be included in the construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

In still another embodiment of a drug screening, a two hybrid assay can be generated with a VSMC protein and target molecule. Drug dependent inhibition or potentiation of the interaction can be scored. The two hybrid assay formats described in the art can be readily adaoted for such drug screening embodiments. See, for example, U.S. Pat. Nos. 5,283,317, 5,580,736 and 5,695,941; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696)

In addition to small molecules which may be identified, e.g., by the drug screening assays described above, other agents capable of modulating smooth muscle cell differentiation may include peptide domains (fragments) of the VSMC protein, as well as mutants of the molecular regulators. A “mutant” as used herein refers to a peptide having an amino acid sequence which differs from that of the naturally occurring peptide or protein by at least one amino acid. Mutants may have the same biological and immunological activity as the naturally occurring protein. However, the biological or immunological activity of mutants may differ or be lacking. For example, a protein mutant may act as an agonist, antagonist (competitive or non-competitive), or partial agonist of the function of the naturally occurring protein.

For example, homologs of the VSMC proteins (both agonist and antagonist forms) can be generated using, for example, alanine scanning mutagenesis and the like (Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085), by linker scanning mutagenesis (Gustin et al. (1993) Virology 193:653-660; Brown et al. (1992) Mol. Cell Biol. 12:2644-2652; McKnight et al. (1982) Science 232:316); by saturation mutagenesis (Meyers et al. (1986) Science 232:613); by PCR mutagenesis (Leung et al. (1989) Method Cell Mol Biol 1:11-19); or by random mutagenesis (Miller et al. (1992) A Short Course in Bacterial Genetics, CSHL Press, Cold Spring Harbor, N.Y.; and Greener et al. (1994) Strategies in Mol Biol 7:32-34). Linker scanning matagenesis, particularly in a combinatorial setting, is on attractive method for identifying truncated (such as constitutively active or dominant negative) forms of a VSMC protein.

The invention also contemplates the reduction of the subject VSMC protein to generate mimetics, e.g. peptide or non-peptide agents, which are able intefere with., or mimic, the effect of the authentic VSMC protein on the growth state of smooth muscle cells. Such peptidomimetics can act as drugs for the modulation of smooth cell differentiation.

Peptidomimetics are commonly understood in the pharmaceutical industry to include non-peptide drugs having properties analogous to those of the mimicked peptide. The principles and practices of peptidomimetic design are known in the art and are described, for example, in Fauchere, Adv. Drug Res. 15:29 (1986); and Evans et al., J. Med. Chem. 30:1229 (1987). Peptidomimetics which bear structural similarity to therapeutically useful peptides may be used to produce an equivalent therapeutic or prophylactic effect. Typically, such peptidomimetics have one or more peptide linkages optionally replaced by a linkage which may convert desirable properties such as resistance to chemical breakdown in vivo. These linkages may include —CH₂NH—, —CH₂S—, —CH₂—CH₂—, —CH═CH—, —COCH₂—, —CH(OH)CH₂—, and —CH₂SO—. Peptidomimetics may exhibit enhanced pharmacological properties (biological half life, absorption rates, etc.), different specificity, increased stability, production economies, lessened antigenicity and the like which makes their use as therapeutics particularly desirable.

Such mutagenic techniques as described above are also particularly useful for mapping the determinants of a VSMC proteins which participate in protein-protein interactions involved in, for example, binding of the subject LTBP-1 protein (described infra) to a TGFβ. To illustrate, the critical residues of a VSMC protein which are involved in molecular recognition of other cellular proteins (or nucleic acid) can be determined and used to generate peptidomimetics which maintain at least a portion of that binding activity. By employing, for example, scanning mutagenesis to map the amino acid residues involved in binding, peptidomimetic compounds (e.g. diazepine or isoquinoline derivatives) can be generated which mimic those residues in binding to the kinase. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J. Med. Chem: 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Modulation of smooth muscle cell differentiation according to the invention includes methods employing specific antisense polynucleotides complimentary to all or part of the nucleotide sequences encoding peptide domains comprising the protein or antisense polynucleotides complimentary to all or part of the 3′ or 5′ noncoding regions of a VSMC protein. Such complimentary antisense polynucleotides may include nucleotide additions, deletions, substitutions and transpositions, providing that specific hybridization to the target sequence persists.

As used herein, “antisense” therapy refers to administration or in situ generation of oligonucleotide probes or their derivatives which specifically hybridize (e.g. bind) under cellular conditions with cellular mRNA and/or genomic DNA encoding a VSMC protein. The hybridization should inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

Soluble antisense RNA or DNA oligonucleotides which can hybridize specifically to mRNA species encoding proteins comprising the molecular regulators, and which prevent transcription of the mRNA species and/or translation of the encoded polypeptide are contemplated as complimentary antisense polynucleotides according to the invention.

An antisense construct of the present invention can be delivered, for example, as an expression plasmid which, when transcribed in the cell, produces RNA which is complementary to at least a unique portion of the target cellular mRNA. Alternatively, the antisense construct is an oligonucleotide probe which is generated ex vivo and which, when introduced into the cell causes inhibition of expression by hybridizing with the mRNA and/or genomic sequences of a target VSMC gene. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Several considerations should be taken into account when constructing antisense oligonucleotides for the use in the methods of the invention: (1) oligos should have a GC content of 50% or more; (2) avoid sequences with stretches of 3 or more G's; and (3) oligonucleotides should not be longer than 25-26 mers. When testing an antisense oligonucleotide, a mismatched control can be constructed. The controls can be generated by reversing the sequence order of the corresponding antisense oligonucleotide in order to conserve the same ratio of bases.

Computer-aided molecular modeling of the VSMC proteins can be used to study three-dimensional structures using computer visualization techniques. Novel designs of low molecular weight inhibitors or oligopeptides can then be analyzed for selective inhibition. Descriptions of targeted drug design can be found in Kuntz, “Structure-Based Strategies for Drug Design and Discovery,” Science 257:1078-1082 (1992) and Dixon, “Computer-Aided Drug Design: Getting the Best Results,” Trends in Biotechnology, 10:357-363 (1992). Specific applications of the binding of inhibitors to targets using computer modeling have been described in Piper et al., “Studies Aided by Molecular Graphics of Effects of Structural Modifications on the Binding of Antifolate Inhibitors to Human Dihydrofolate Reductase,” Proc. Am. Assoc. Cancer Res. Annual Meetin, 33:412 (1992); Hibert et al., “Receptor 3D-Models and Drug Design,” Therapie (Paris), 46:445-451 (1991)(serotonin receptor recognition sites). Computer programs that can be used to conduct three-dimensional molecular modeling are described in Klopman, “Multicase 1: A Hierarchical Computer Automated Structure Evaluation Program,” Quantitative Structure-Activity Relationships, 11:176-184 (1992); Pastor et al., “The Edisdar Programs Rational Drug Series Design,” Quantitative Structure-Activity Relationships, 10:350-358 (1991); Bolis et al., “A Machine Learning Approach to Computer-Aided Molecular Design,” J. Computer Aided Molecular Design, 5:617-628 (1991); and Lawrence and Davis, “CLIX: A Search Algorithm for Finding Novel Ligands Capable of Binding Proteins of Known Three-Dimensional Structure,” Proteins Structure Functional Genetics, 12:31-41 (1992).

In still other embodiments, low molecular weight. inhibitors specific for the molecular regulators can be predicted by molecular modeling and synthesized by standard organic chemistry techniques. Computer modeling can identify oligopeptides which enhance the smooth muscle cell differentiation or block their dedifferentiation. Techniques for producing the identified oligopeptides are well known and can proceed by organic synthesis of amino acids, by genetic engineering techniques, or by PCR based amplification. Silverman, The Organic Chemistry of Drug Design and Drug Action, Academic Press (1992). The inhibitors of this invention can be identified as those inhibitors that selectively inhibit the smooth muscle cell dedifferentiation.

In preferred embodiments, the drug screening assay is carried out with a VSMC protein which is selectively expressed in smooth muscle cells, e.g., relative to other non-myocytic tissues, and even more preferably, relative to other non-myocytic and myocytic tissues. For example, in certain preferred embodiments, the VSMC protein selected as a candidate for drug development will be a protein which is either not expressed in other muscle cells (such as skeletal muscle), or is not required in those cells for growth or differentiation. However, it will also be evident to those skilled in the art that selectivity can also be provided by the mode of administration of a drug identified in the subject assays. For instance, delivery of the drug by catheter, or by injection into the pericardial space, and provide selectivity for vascular smooth muscle cells relative to, e.g., skeletal muscle or other tissues.

In certain embodiments, therapeutic agents of the invention are selected to inhibit a cellular activity of a vascular smooth muscle cell, e.g., proliferation, migration, increase in cell volume, increase in extracellular matrix synthesis (e.g., collagens, proteoglycans, and the like), or secretion of extracellular matrix materials by the cell. Preferably, the therapeutic agent acts either: a) as a “cytostatic agent” to prevent or delay cell division in proliferating cells by inhibiting replication of DNA (e.g., a drug such as adriamycin, staurosporin, tamoxifen or the like), or by inhibiting spindle fiber formation (e.g., a drug such as colchicine) and the like; or b) as an inhibitor of migration of vascular smooth muscle cells from the medial wall into the intima, e.g., an “anti-migratory agent” ; or c) as an inhibitor of the intracellular increase in cell volume (i.e., the tissue volume occupied by a cell; a “cytoskeletal inhibitor” or “metabolic inhibitor”); or d) as an inhibitor that blocks cellular protein synthesis and/or secretion or organization of extracellular matrix (i.e., an “anti-matrix agent”).

The VSMC therapeutic may be administered alone, or conjointly with: (1) therapeutic agents that alter cellular metabolism or are inhibitors of protein synthesis, cellular proliferation, or cell migration; (2) microtubule and microfilament inhibitors that affect morphology or increases in cell volume; and/or (3) inhibitors of extracellular matrix synthesis or secretion

Latent TGFβ Binding Protein

In one embodiment, the subject VSMC therapeutic is identified by its ability to modulate the activity of a latent TGFβ binding protein (LTBP), preferably LTBP-1. Transforming growth factor-beta (TGFβ) is secreted as a latent, high molecular weight complex, which is composed of TGFβ, a latency associated peptide (LAP) and a latent TGFβ binding protein (LTBP). LTBP, a component of the extracellular matrix (ECM) of various tissues, is important for the secretion of TGFP and, furthermore, for the storage of TGFβ in ECM. Inhibition of the activity of LTBP can be used as part of a treatment intended to inhibit proliferation and/or migration of smooth muscle cells, e.g., to treat or inhibit the formation of a luminal intima.

Previous studies have shown that LTBP-1 binds the small latent TGFβ1 complex through a disulfide bond between an 8-cysteine structural motif of LTBP-1 (a “TGF-bp repeat”) and the propeptide dimer of LAP. Accordingly, in certain embodiments, the invention contemplates the identification of VSMC therapeutics which inhibit the interaction of LTBP-1 with the small latent TGFβ1 complex, e.g., by competive or non-competitive binding to LAP or LTBP-1. Exemplary VSMC therapeutics can be, e.g., small organic molecules which inhibit the formation of LTBP-1-containing TGFβ complexes, as well as polypeptide, peptide fragments and peptidomimetics of LTBP-1 which competively bind the small latent TGFβ1 complex and inhibit binding of the native LTBP.

The proteolytic cleavage of LTBP is believed to be the prerequisite for the activation of TGF-beta. Thus, in another embodiment, it is contemplated that VSMC therapeutic can be identified which inhibit the proteolysis of LTBP, e.g., protease inhibitors.

In other embodiment, antisense can be used to inhibit expression of LTBP-1.

Sources for generating recombinant forms of LTBP-1, LAP and TGFβ are known in the art. For example, the sequence for human LTBP-1 is provided at SWISS-PROT accession P22064, and Kanzaki et al. (1990) Cell 61:1051-1061.

Integrin-Linked Kinase

Another differentially expressed gene identifed herein encodes an integrin-linked kinase (ILK). Thus, in another embodiment, the subject VSMC therapeutic is identified by its ability to modulate the activity of an ILK. The interaction of cells with the extracellular matrix regulates cell shape, motility, growth, survival, differentiation and gene expression, in part through integrin-mediated signal transduction. Utilizing the assay described herein, ILK was cloned on the basis of being upregulated during the development of smooth muscle cells. ILK is an ankyrin repeat containing serine-threonine protein kinase that can interact directly with, and phosphorylate, the cytoplasmic domains of the β1 and β3 integrin subunits and whose kinase activity is modulated by cell- extracellular matrix interactions. Overexpression of ILK disrupted epithelial cell architecture and inhibited adhesion to integrin substrates, while inducing anchorage- independent growth. Indeed, ILK-overexpressing epithelial cells readily formed tumors in nude mice. Based on our observations herein, we suggest a novel critical role of this kinase in smooth muscle cell growth, cell survival, and tumorigenesis.

Integrins are heterodimeric integral plasma membrane proteins containing extracellular, transmembrane, and cytoplasmic domains. The cytoplasmic domains of integrins are required for the transduction of this bidirectional information, and have recently been shown to participate in direct interactions with ILK. The present invention contemplates that VSMC therapeutics can be identified which antagonize the role of ILK in regulation of smooth muscle cells. For instance, compounds can be identified which inhibit the phosphorylation of integrins and other substrates by ILK. To illustrate, the subject drug screening assays can be used to identify mechanistic or other competitive inhibitors of the kinase activity of ILK, e.g., small molecule inhibitors. Likewise, small molecules (including peptidyl fragments of ILK or an integrin) can be identified by their ability to inhibit interaction of ILK with its substrates.

Moreover, overexpression of ILK in intestinal epithelial cells results in translocation of β-catenin to the nucleus, formation of a complex between β-catenin and the high mobility group transcription factor, LEF-1, and transcriptional activation by this LEF-1/β-catenin complex. Thus, inhibition of ILK activity in smooth muscle cells may also utilize inhibitors of the LEF-1/β-catenin signaling pathway.

As above, antisense can be used to inhibit expression of ILK.

Conversely, constitutively active forms of ILK (see, e.g., Novak et al. (1998) PNAS 95:4374) can expressed to potentiate the activity of ILK in smooth muscle cells.

Decorin

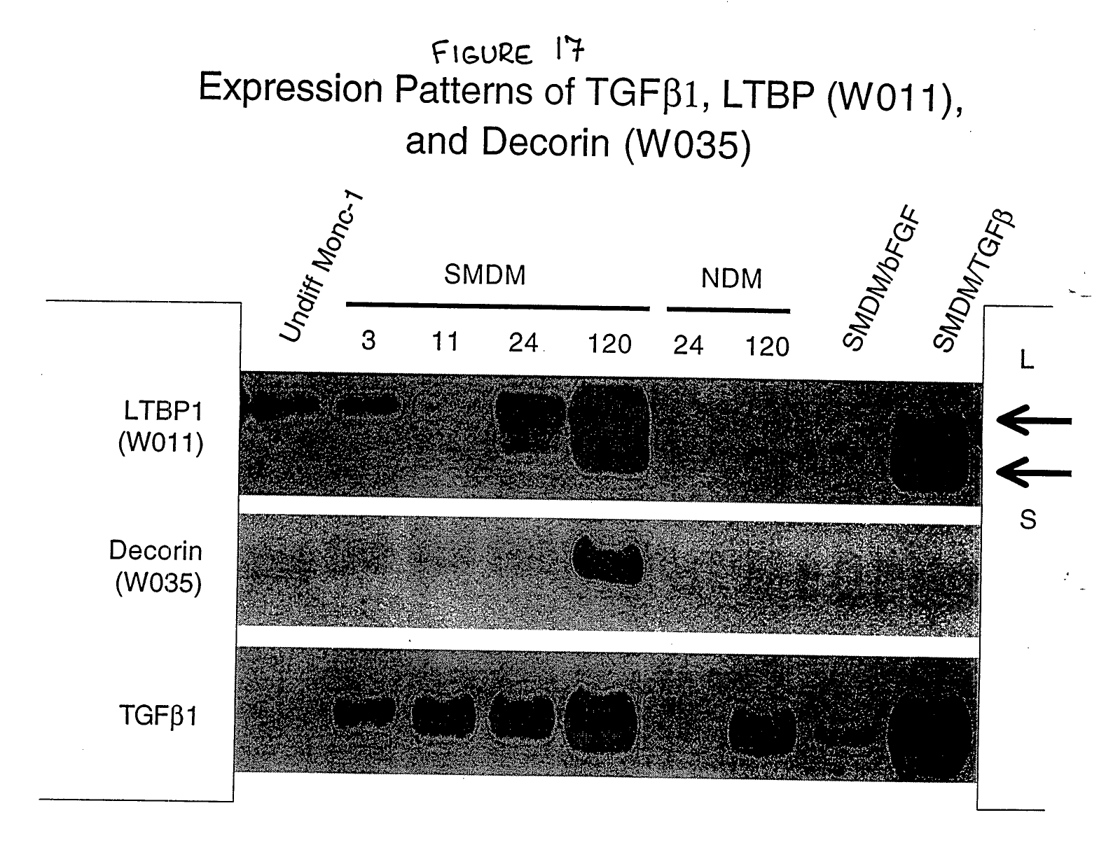

Another differentially expressed nodal regulator is the decorin protein. FIG. 17 illustrates the selective expression pattern of TGFβ, LTBP and Decorin. TGFβ specifically binds to members of the decorin family of proteoglycans such as decorin, biglycan, fibromodulin and lumican. Given the identification of LTBP-1, the role of TGFβ as a promoter of alternative neural crest cell fate, the TGFβ inhibitory activity of decorin is a contemplated drug screening target for agents which modulate the growth state of smooth muscle cells.

Decorin, also known as PG-II or PG-40, is a small proteoglycan. Its core protein has a molecular weight of about 40,000 daltons. The core has been sequenced and it is known to carry a single glycosaminoglycan chain of the chondroitin sulfate/dermatan sulfate type. Most of the core protein of decorin is characterized by the presence of a leucine-rich repeat (LRR) of about 24 amino acids.

Decorin has been used to prevent TGFβ-induced cell proliferation and extracellular matrix production. Decorin is therefore useful for reducing or preventing pathologies caused by TGF beta-regulated activity. Methods for expressing and purifying human recombinant decorin are known in the art, for example, as described in PCT application WO 90/00194 and U.S. Pat. No. 5,763,276. U.S. Pat. No. 5,705,609 describes inhibitory fragments of decorin which may be useful in the treatment methods of the present invention.

In certain embodiments, the subject drug screening assays are used to identify agents which disrupt the interaction of decorin with TGFβ, or alternatively, which mimic or potentiate the interaction.

Caspase

Another potential drug target detected in the subject SMC differentiation assay is a thiol protein, Caspase-4 (GenBank P49662; see also Faucheu et al. (1995) EMBO J. 14:1914). Caspase-4 is a so-called “death protease”, which is activated by caspase-8 (by proteolysis), and activates caspase-3 (also be proteolysis). The protease activities of any of these enzymes, are therefore, targets for drug discovery in the subject screening assays.

Moreover, the active enzyme is a heterodimer. The two subunits are derived from the precursor sequence by an autocatalytic mechanism or by cleavage by caspase-8. The interaction of the subunits can be inhibited, for example, by a small molecule identified in a drug screening assay of the present invention.

Finally, the caspase enzymes are understood to interact with CED proteins. In particular, caspase-4 interacts with the mammalian homolog of CED-4, Apaf-1. This interaction is also a potential target for drug discovery.

Torsin

Another potential target for therapeutic intervention is the product of the human homolog of the early-onset torsion dystonia gene (DYT1), or torsina, which encodes an ATP-binding protein. It resembles a class of proteins that protects cells from stress and trauma, the heat-shock proteins/proteases. Ozelius et al. (1997) Nat Genet 17:40-48. Techniques for developing compounds which inhibit or potentiate the activity of torsinA can be adapated from the art, such as U.S. Pat. No. 5,750,119 which describes drug screening assays based on related heat shock protein complexes.

cctζ

The chaperonin-containing TCP-1 complex (CCT) is a heteromeric particle composed of multiple different subunits. We have identified a subunit, cctζ, which is selectively upregulated during differentiation. In other instance, tissue-specific subunits of TCP-1 have been reported. See, for example, Kubota et al. (1997) FEBS Lett 402:53-56. The cctζ subunit may have specific functions in the folding of SMC proteins and for interactions with SMC molecular chaperones.

In one embodiment, the subject drug screening assay can be adapted for detection of compounds which inhibit the interaction of ccζ with the TCP complex.

Prothymosin Alpha

Prothymosin-α is a small, highly acidic, abundant, nuclear, mammalian protein which is essential for cell growth is known to be covalently attached to a small cytoplasmic RNA in mammalian cells. Mutational analysis of human Prothymosin-α reveals a bipartite nuclear localization signal. Its phosphorylation status is correlated with proliferative activity. In one embodiment, the kinase, or antagonistic phosphatase, involved in regulating the phosphorylation status of Prothymosin-α are potential targets for inhibitors useful as VSMC therapeutics.

Prothymosin-α binds histones in vitro and shows activity in nucleosome assembly assay. This interaction is also a potential target for development of inhibitors useful as VSMC therapeutics.

Lim-Kinase

LIM-kinase 1 (LIMK1) and LIM-kinase 2 (LIMK2) are members of a serine/threonine kinase subfamily with structural features composed of N-terminal two LIM domains, an internal PDZ-like domain, and a C-terminal protein kinase domain. In certain embodiments, the subject assays are generated to find inhibitors of the kinase activity of LIM-kinase 2b.

LIM domains, Cys-rich motifs containing approximately 50 amino acids found in a variety of proteins, are proposed to direct protein-protein interactions. Thus, the assays can be used to identify agents which inhibit interaction of the LIM domains with, e.g., LIM domain-binding proteins, as well as with DNA.

In other embodiments, the assays are designed to identify activators of LIM-kinase 2b, such as constitutively active forms.

Cca

A cDNA fragment, named ccal (confluent 3Y1 cell-associated 1), was previously isolated on the basis of preferential accumulation of the corresponding mRNA in growth-arrested confluent but not in growing subconfluent rat 3Y1 cells. GenBank Accession AB000215.

Interferon Aactivatable Protein

Another gene which is differentially expressed in the SMC culture is an interferon activatable protein, p204, GenBank Accession M31419. See also Choubey et al. (1989) J Biol Chem 264:17182. Like pRB and p107, p204 is a potent growth inhibitor in sensitive cells. It is also a phosphoprotein, so the drug screening assay can be designed to detect agents which inhibit or potentiate the phosphorylation state of p204.

Internexin

Another protein identified in the screen is a cytoskeletal element, α-internexin (GenBank L27220, see also Chien et al. (1994) Gene 149:289). The interactions of α-internexin with other cyctoskeletal elements, such as intermediate filament elements, can be a target for drug development.

Desmoyokin/AHNAK

Another protein identified in the SMC screen is Desmoyokin, or AHNAK, gene product. GenBank X74818 and X65157. Desmoyokin undergoes calcium-mediated sequestration from a diffuse pattern at low calcium to localization at the cell boundaries at high calcium concentrations. TPA also induced translocation of the desmoyokin protein. Selective PKC inhibitors completely inhibited the calcium-induced translocation of the desmoyokin protein. Moreover, the desmoyokin protein undergoes calcium-induced phosphorylation, possibly by PKC. The abundance of the protein increases appreciably when cells withdraw from the division cycle, e.g., in response to differentiation. By contrast, the amount of phosphorylation appears to diminish under those conditions.

Thus, the translocation of the desmoyokin protein is a target for drug screening. Likewise, the rate of phosphorylation (or dephosphorylation) of the protein can be the target for drug identification.

TSC-36 (TGF Inducible Protein)

Still another target identified in the subject SMC differentiation assay is the TGFβ-inducible protein, TSC-36. See GenBank M91380, and Shibanuma et al. (1993) Eur J Biochem 217:13. The amino acid sequence of TSC-36 protein was found to have some similarity with follistatin, an activin-binding protein, and a limited similarity with the secreted protein rich in cysteine (SPARC). The biological activity of TSC-36, e.g., as a paracrine or autocrine factor, can be the target for drug development.

T-Cell-Activating Protein (TAP)

Yet another target identified by the subject SMC assay is the T-cell-activating protein TAP, which is a phosphatidylinositol-anchored glycoprotein. See GenBank J03636 and Reiser et al. (1988) Proc Natl Acad Sci U S A 85:2255. The interaction of the TAP with extracellular components, the transduction of signal across the cell membrane, and the addition of the phosphatidylinositol anchor are each potential targets for developing small molecule inhibitors or agonists of TAP function in SMC.

Transcobalamin

Another target for the subject drug screening assays is the transcobalamin, TCII. See GenBank AF047576 and AF090686. Transcobalamin II (TCII) is a plasma protein that binds vitamin B 12 (cobalamin; Cbl) and facilitates the cellular uptake of the vitamin by receptor-mediated endocytosis.

Fos-Related Antigen

Fos-related antigen-2 (fra-2) was also identifed as being upregulated in the SMC assay. That gene, see GenBank U18913 and Baler et al. (1995) J Biol Chem 270:27319, is a member of the Fos family of transcription factor. The interaction of fra-2 with other transcriptional components, e.g., to form AP-1 complexes or CREB complexes, as well as its interaction with DNA (as a monomer or part of a transcriptional complex) are potential targets for development of inhibitors of fra-2 activity in SMC.

Epididymal Secretory Protein E1 Precursor (HE1)

The subject SMC differentiaion assay also identified HE1 as a potential drug screening target. HE1 is a major secretory protein of the human epididymis. See GenBank Q15668 and Krull et al. (1993) Mol Reprod Dev 34:16. Kirchhoff et al. (1996) Biol Reprod 54:847.

Ubiquitin Carboxyl-Terminal Hydrolase 12 (UCH-12)

Still another potential drug screening target is ubiquitin carboxyl-terminal hydrolase 12. Ubiquitin is expressed in eukaryotic cells as precursors, fused via its carboxyl terminus either to other ubiquitin sequences in linear polyubiquitin arrays or to specific ribosomal proteins. In some of the polyubiquitin fusions a single amino acid (e.g., valine in humans) is attached to the carboxyl terminus. These gene products are rapidly cleaved by ubiquitin carboxyl-terminal hydrolase (UCH) enzymes. Thus, the ubiquitin- dependent proteolytic activity of UCH-12, alone or as a polysome, can be targeted for inhibition in SMC

Thyrptropin Releasing Hormone

Thyrotropin-releasing hormone (TRH), was also identified as being upregulated in differentiating SMC's. TRH binds to a G protein-coupled receptor (TRH-R). The interaction of TRH with the receptor, and the subsequent intracellular signal transduction by the TRH receptor, are each potential targets for agonists or antagonists of TRH in smooth muscle cells.

It will be apparent to those skilled in the art that the subject drug screening assays can be carried out using the clones described above, or homologs thereof (e.g., which are encoded by nucleic acids which hybridize under stringeny conditions to the enumerated sequence), or fragments thereof which retain the biological activity for which an agonist or antagonist is sougth. In preferred embodiment, the assay is carried out using a human homolog of the targeted protein.

D. Methods for Treating or Preventing Arteriosclerosis by Inhibiting or Regulating the Activity of Smooth Muscle Cell Differentiation.

Another aspect of the present invention relates to a method of inducing and/or maintaining a differentiated state, enhancing survival, and/or inhibiting (or alternatively potentiating) proliferation of a smooth muscle cell, by contacting the cells with an agent which modulates the activity of a VSMC protein. A “VSMC therapeutic,” whether inhibitory or potentiating with respect to modulating the activity of a VSMC protein, can be, as appropriate, any of the preparations described herein, including isolated VSMC proteins (including both agonist and antagonist forms), gene therapy constructs, antisense molecules, peptidomimetics, or agents identified in the drug assays provided herein.

Inappropriate vascular smooth muscle proliferation is an integral component of the pathophysiology of several clinically important forms of vascular disease. Proliferation and migration of vascular smooth muscle cells are an important mechanism of the genesis of the atherosclerotic plaques that cause heart attacks, strokes or peripheral vascular disease (N Eng J Med;314:488-500, 1986). Restenosis after treatment of atherosclerotic vascular lesions with percutaneous transluminal angioplasty is a common adverse clinical outcome of this procedure. Restenosis involves a proliferative response of vascular smooth muscle at the site of the injury (J Am Coll Cardiol;6:369-375, 1985). Finally vascular smooth muscle proliferation has been proposed as an important component in the genesis of other forms of vascular injury or obstruction including closure of surgical bypass tracts (J Vascular Research;29:405-409, 1992), irradiation injury to the vasculature (Am Coll Cardiol;19:1106-1113, 1992), systemic hypertension (J Hypertension;12:163-172, 1994) and neonatal or primary pulmonary hypertension (J Clin Invest;96:273-281, 1995 and Circulation;42:1163-1184, 1970).

In certain embodiments, antiproliferative forms of the VSMC therapeutics identified by the subject drug screening assays can be used to inhibit proliferation of smooth muscle cells, e.g., in vitro or in vivo. For instance, many pathological conditions have been found to be associated with smooth muscle cell proliferation for which treatment can be carried out with anti-proliferative agents identified by the the methods of the present invention. Such conditions include restenosis, atherosclerosis, coronary heart disease, thrombosis, myocardial infarction, stroke, smooth muscle neoplasms such as leiomyoma and leiomyosarcoma of the bowel and uterus and uterine fibroid or fibroma.

For example, percutaneous transluminal coronary angioplasty (PTCA) is widely used as the primary treatment modality in many patients with coronary artery disease. PTCA can relieve myocardial ischemia in patients with coronary artery disease by reducing lumen obstruction and improving coronary flow. The use of this surgical procedure has grown rapidly, with over 500,000 PTCAs being performed per year. Stenosis following PTCA remains a significant problem, with from 25% to 35% of the patients developing restenosis within 1 to 3 months. Restenosis results in significant morbidity and mortality and frequently necessitates further interventions such as repeat angioplasty or coronary bypass surgery.

In one embodiment, an anti-proliferative VSMC therapeutic can be administered to inhibit stenosis due to proliferation of vascular smooth muscle cells following, for example, traumatic injury to vessels rendered during vascular surgery. The therapeutic conjugates and dosage forms of the invention are useful for inhibiting the activity of vascular smooth muscle cells, e.g., for reducing, delaying, or eliminating stenosis following angioplasty. As used herein the term “reducing” means decreasing the intimal thickening that results from stimulation of smooth muscle cell proliferation following angioplasty, either in an animal model or in man. “Delaying” means delaying the time until onset of visible intimal hyperplasia (e.g., observed histologically or by angiographic examination) following angioplasty and may also be accompanied by “reduced” restenosis. “Eliminating” restenosis following angioplasty means completely “reducing” and/or completely “delaying” intimal hyperplasia in a patient to an extent which makes it no longer necessary to surgically intervene, i.e., to re-establish a suitable blood flow through the vessel by repeat angioplasty, atheroectomy, or coronary artery bypass surgery. The effects of reducing, delaying, or eliminating stenosis may be determined by methods routine to those skilled in the art including, but not limited to, angiography, ultrasonic evaluation, fluoroscopic imaging, fiber optic endoscopic examination or biopsy and histology. The therapeutic conjugates of the invention achieve these advantageous effects by specifically binding to the cellular membranes of smooth muscle cells and pericytes.

In another embodiment, the invention provides a method for treating or preventing arteriosclerosis. An effective amount of an agent which inhibits the smooth muscle cell dedifferentiation or enhance smooth muscle differentiation is administered to animal modes of arteriosclerosis such as balloon injured carotid arteries in rats or apoE−/− mice that develop atherosclerotic plaques similar to the human lesions. The molecules that show a beneficial effect will be used to treat patients. Administration may be periodic or continuous as desired for the prevention or treatment of arteriosclerosis.

Still another aspect of the present invention relates to therapeutic modalities for maintaining an expanded luminal volume following angioplasty or other vessel trauma. One embodiment of this aspect of the present invention involves administration of a therapeutic agent capable of inhibiting the ability of vascular smooth muscle cells to contract. Exemplary agents useful in the practice of this aspect of the present invention are those capable of causing a traumatized artery to lose vascular tone, such that normal vascular hydrostatic pressure (i.e., blood pressure) expands the flaccid vessel to or near to its maximal physiological diameter. Loss of vascular tone may be caused by agents that interfere with the formation or function of contractile proteins (e.g., actin, myosin, tropomyosin, caldesmon, calponin or the like). This interference can occur directly or indirectly through, for example, inhibition of calcium modulation, phosphorylation or other metabolic pathways implicated in contraction of vascular smooth muscle cells.

### (iv) PHARMACEUTICAL PREPARATIONS OF IDENTIFIED AGENTS

After identifying certain test SLEs as selectively antiproliferative, the practitioner of the subject assay will continue to test the efficacy and specificity of the selected SLEs both in vitro and in vivo. Whether for subsequent in vivo testing, or for administration to an animal as an approved drug, antiproliferative peptides identified in the subject assay, or peptidomimetics thereof, can be formulated in pharmaceutical preparations for in vivo administration to an animal, preferably a human. Likewise, antisense SLEs can be generated as non-hydrolizable analogs (e.g., resistant to nuclease degradation) and formulated for direct administration, or, as appropriate, provided in the form of an expression vector, such as for gene therapy, which produces the antisense molecule as a transcript. SLEs which are active as polypeptides can also be provided in the form of an expression vector for use, e.g., in gene therapy.

The peptides, proteins and antisense selected in the subject assay, or gene therapy vectors encoding such molecules, may accordingly be formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the compound, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”. Based on the above, such pharmaceutical formulations include, although not exclusively, solutions or freeze-dried powders of the compound in association with one or more pharmaceutically acceptable vehicles or diluents, and contained in buffered media at a suitable pH and isosmotic with physiological fluids. In preferred embodiment, the SLE compound can be disposed in a sterile preparation for topical and/or systemic administration. In the case of freeze-dried preparations, supporting excipients such as, but not exclusively, mannitol or glycine may be used and appropriate buffered solutions of the desired volume will be provided so as to obtain adequate isotonic buffered solutions of the desired pH. Similar solutions may also be used for the pharmaceutical compositions of compounds in isotonic solutions of the desired volume and include, but not exclusively, the use of buffered saline solutions with phosphate or citrate at suitable concentrations so as to obtain at all times isotonic pharmaceutical preparations of the desired pH, (for example, neutral pH).

### (v) EXEMPLIFICATION

The invention now being generally described, it will be more readily understood by reference to the following examples which are included merely for purposes of illustration of certain aspects and embodiments of the present invention, and are not intended to limit the invention.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include Sambrook, et al., infra; McPherson, Ed., Directed Mutagenesis: A Practical Approach, IL Press, Oxford (1991); Jones, Amino Acid and Peptide Synthesis, Oxford Science Publications, Oxford (1992); Austen and Westwood, Protein Targeting and Secretion, IL Press, Oxford (1991). Any suitable materials and/or methods known to those of skill can be utilized in carrying out the present invention; however, preferred materials and/or methods are described. Materials, reagents and the like to which reference is made in the following description and examples are obtainable from commercial sources, unless otherwise noted.

### EXAMPLE 1

In Vitro System for Differentiating Pluripotent Neural Crest Cells into Smooth Muscle Cells*

The change in vascular smooth muscle cells (SMC) from a differentiated to a dedifferentiated state is the critical phenotypic response that promotes occlusive arteriosclerotic disease. Despite its importance, research into molecular mechanisms regulating smooth muscle differentiation has been hindered by the lack of an in vitro cell differentiation system. This example identifies culture conditions that promote efficient differentiation of Monc-1 pluripotent neural crest cells into SMC. Exclusive Monc-1 to SMC differentiation was indicated by cellular morphology and time-dependent induction of the SMC markers smooth muscle -actin, smooth muscle myosin heavy chain, calponin, SM22, and APEG-1. The activity of the SM22 promoter was low in Monc-1 cells. Differentiation of these cells into SMC caused a 20-30-fold increase in the activity of the wild-type SM22 promoter and that of a hybrid promoter containing three copies of the CArG element. By gel mobility shift analysis, we identified new DNA-protein complexes in nuclear extracts prepared from differentiated Monc-1 cells. One of the new complexes contained serum response factor. This Monc-1 to SMC model should facilitate the identification of nodal regulators of smooth muscle development and differentiation.

Pluripotent neural crest cells can differentiate into neurons, glia, chondrocytes, melanocytes, and SMC. Arterial SMC of the chick ascending and thoracic aorta are of a neural crest origin, and various members of the transforming growth factor-superfamily can instructively promote differentiation of primary cultured neural crest cells into neuronal cells or SMC. Unfortunately, our ability to work with neural crest cells in primary culture has been limited by the difficulty of obtaining quantities sufficient for biochemical and genetic analysis. This problem was solved recently by the generation of an immortalized neural crest cell line, Monc-1, by retroviral transfection of mouse neural crest cells with the v-myc gene.

We hypothesized that Monc-1 cells could be used to develop an in vitro SMC differentiation system. We describe in this report the culture conditions under which Monc-1 cells can be differentiated efficiently into SMC. Exclusive Monc-1 to SMC differentiation was indicated by cellular appearance and induction of the SMC markers smooth muscle-actin, smooth muscle myosin heavy chain, calponin, SM22, and APEG-1. Also, a 20-30-fold increase in the activity of the SMC-specific promoter SM22 coincided with the formation of new DNA-protein complexes during differentiation.

Experimental Procedure

Cell Culture and Reagents—The Monc-1 cell line was kindly provided by David Anderson (Pasadena, Calif.). Monc-1 cells were cultured in the undifferentiated state on fibronectin-coated plates in an L-15 CO₂-based medium supplemented with chick embryo extract, hereafter referred to as complete medium, as described by Stemple and Anderson (Stemple and Anderson, (1992) Cell 71, 973-985). Differentiation down the neuronal and glial pathways was performed on plates coated sequentially with polylysine (0.5 mg/ml) and fibronectin (0.25 mg/ml) in complete medium supplemented with 10% fetal bovine serum (Hyclone, Logan, Utah) plus 5 mM forskolin (Sigma, St. Louis, Mo.) as described in Sommer, et al. (1995) Neuron 15, 1245-1258. Smooth muscle cell differentiation was induced by application of the media components listed in Table 1 supplemented with 10% fetal bovine serum, penicillin (100 units/ml), streptomycin (100 μg/ml), and 25 mM Hepes (pH 7.4), hereafter referred to as smooth muscle cell differentiation medium (SMDM).

RNA Extraction and Northern Analysis—Total RNA from cultured cells was prepared by guanidinium isothiocyanate extraction and centrifugation through cesium chloride according to the procedures described in Sambrook, et al., infra. Total RNA was fractionated on a 1.3% formaldehyde-agarose gel and transferred to nitrocellulose filters, which were hybridized with the appropriate, randomly primed, ³²P-labeled probe. The hybridized filters were washed in 30 mM sodium chloride, 3 mM sodium citrate, and 0.1% sodium dodecyl sulfate at 55° C. Autoradiography was performed with Kodak XAR film at 80° C. To correct for differences in loading, the filters were washed in a 50% formamide solution at 80° C. and rehybridized with a radiolabeled 18S rRNA oligonucleotide probe (Yoshizumi, et al. (1992) J. Biol. Chem. 267, 9467-9469). The filters were scanned and radioactivity was measured on a PhosphorImager running the Imagequant software (Molecular Dynamics, Sunnyvale, Cailf.). The smooth muscle α-actin was provided by J. Lessard (Cincinnati, Ohio); the AT2 cDNA was provided by A. D. Strosberg (Paris, France). The calponin cDNA was isolated from a mouse aortic cDNA library. The glial fibrillary acidic protein cDNA was isolated by reverse transcriptase polymerase chain reaction from brain RNA using the forward primer 5′AGCCAAGGAGCCCACCAAACT3′ and the reverse primer 5′TTACCACGATGTTCCTCTTGA3′. cDNA authenticity was confirmed by the dideoxy chain termination method.

mRNAs for the smooth muscle myosin heavy chain isoforms SM1 and SM2 were detected by reverse transcription PCR with primers designed from the mouse SM1 and SM2 cDNAs (GenBank accession numbers D85923 and D85924). The forward primer 5′ AGGAAACACCAAGGTCAAGCA 3′ and the reverse primer 5′ GGGACTGTACCACAGGTTAG 3′ were used to amplify a 324 base pair SM1 fragment and a 363 base pair (alternatively spliced) SM2 fragment. To control for efficiency of reverse transcription, an aliquot of template cDNA was analyzed by PCR with a forward primer (5′ TGAAGGTCGGTGTGAACGGATTTGGC 3′) and a reverse primer (5′ CATGTAGGCCATGAGGTCCACCAC 3′) designed from the mouse glyceraldehyde-3-phosphate dehydrogenase cDNA sequence.

Immunocytochemistry—Monc-1 cells were grown on glass slides coated with fibronectin or fibronectin plus polylysine in the appropriate medium (see Cell Culture and Reagents). Immunostaining for smooth muscle ax-actin, calponin, glial fibrillary acidic protein, and peripherin was performed as described in Shah, et al. (1996) Cell 85, 331-343 and Yoshizumi, et al. (1995) J. Clin. Invest. 95, 2275-2280. Proteins from undifferentiated and differentiated Monc-1 cells and mouse aortas were prepared according to standard procedures (Sambrook, et al. (1989) Molecular Cloning: A Laboratory Manual 2d ed. Cold Spring Harbor Laboratory Press, Plainview, N.Y.) with minor modifications. Proteins were resolved on 5% sodium dodecyl sulfate-polyacrylamide gels (Laemmli (1970) Nature 227, 680-685), transferred electrophoretically to nitrocelluolose membranes (Schleicher and Scheull, Keene, N.H.), and incubated with a rabbit anti-smooth muscle myosin heavy chain antibody (Groschel-Stewart, et al. (1976) Histochemistry 46, 229-236) diluted 1:5000, followed by incubation with a horseradish peroxidase-conjugated goat anti-rabbit antibody diluted 1:4000. Membranes were processed with an enhanced chemiluminescence reagent (Pierce) and exposed to film.

Transfection and Luciferase Assays—A 1.4 kb fragment of the SM22α promoter was obtained by polymerase chain reaction using mouse genomic DNA and the following primers: forward 5′CAGTGGCTGGAAAGCAAGAGC3′ and reverse 5′GGGCTGGGGCAGACGGGC3′. The promoter fragment was subcloned by blunt-end ligation into the XhoI site of the PGL2 basic vector (Promega, Madison, Wis.). Monc-1 cells were transfected transiently by electroporation as described in Kho, et al. (1997) J. Biol. Chem. 272, 3845-3851, with minor modification. In brief, Monc-1 cells were resuspended in PBS and placed in electroporation cuvettes (BTX, San Diego, Calif.) at a final concentration of 1×10⁶ cells. Plasmid DNA was added and electroporated with the Bio-Rad gene pulser at 0.25 V, 500 μFD. Cells were then applied to 60-mm fibronectin-coated plates and placed in complete medium or SMDM. Cell extracts were prepared 48-72 hours after transfection, and luciferase and β-galactosidase assays were performed as described in Braiser, et al. (1989) Biotechniques 7, 1116-1122 and Lee, et al. (1990) J. Biol. Chem. 265, 10446-10450. Each construct was transfected at least six times. Data for each construct are presented as the mean±standard error.

Electrophoretic Mobility Shift Analysis—Nuclear extracts were prepared according to the method of Ritzenthaler et al. (1991) Biochem. J. 280, 157-162. Cells were washed in cold PBS and lysed in nuclear lysis buffer (10 mM Tris, pH 7.6, 10 mM NaCl, 3 mM MgCl₂, 0.5% NP-40). Nuclei were centrifuged at 500 g and then washed with 1 ml of nuclear lysis buffer. Packed nuclei were extracted with buffer (20 mM Hepes, pH 7.9, 350 mM NaCl, 1.5 mM MgCl₂, 0.2 mM EDTA, pH 8.0, 25% glycerol, 0.5 mM dithiothreitol, 5 mg/ml leupeptin, and 1 mg/ml aprontinin) for 20 minutes at 4° C. Samples were centrifuged at 10,000 g for 20 minutes, and supernatants were recovered. Protein concentration was determined with the Bio-Rad protein assay kit (Bio-Rad, Hercules, Calif.) according to manufacturer's instructions by using bovine serum albumin as a standard. Electrophoretic mobility shift analysis was performed as described in Kim, et al. (1997) Mol. Cell. Biol. 17, 2266-2278 and Yoshizumi, et al. (1995) Mol. Cell. Biol. 15, 3266-3272. In brief, double-stranded oligonucleotide probes synthesized according to the sequence of the SM22α CArG element 5′TCGAGACTTGGTGTCTTTCCCCAAATATGGAGCCTGTGTGGAGTG3′ were radiolabeled as described in Yoshizumi, et al. (1995) J. Clin. Invest. 95, 2275-2280. A typical binding reaction mixture contained DNA probe at 20,000 cpm, 1 μg poly(dI-dC) poly(dI-dC), 25 mM Hepes (pH 7.9), 40 mM KCl, 3 mM MgCl₂, 0.1 mM EDTA, 1 mM dithiothreitol, 10% glycerol, and 3 μg of nuclear extract in a final volume of 25 ml. The reaction mixture was incubated at room temperature for 20 min and analyzed by 5% native polyacrylamide gel electrophoresis in 0.25× TBE buffer (22 mM Tris base, 22 mM boric acid, and 0.5 mM EDTA). A 250× excess of specific or nonspecific oligonucleotide was used for competition experiments. For supershift experiments, 1 μl of anti-serum responsive factor antibody (sc-335×, Santa Cruz Biotechnology, Santa Cruz, Calif.) or anti-YY1 antibody (sc-281×, Santa Cruz Biotechnology, Santa Cruz, Calif.) was incubated with nuclear extracts and probes.

Differential Display—The differential display method uses the Hieroglyph system (Genomyx, Foster City, Calif.) in conjunction with the genomyxLR DNA sequencer, which offers reproducibility and the ability to resolve DNA fragments of up to 1000 base pairs.

### EXAMPLE 2

Aortic Carboxypeptidase-Like Protein, a Novel Protein with Discoidin and Carboxypeptidase-Like Domains, Is Up-Regulated during Vascular Smooth Muscle Cell Differentiation

Phenotypic modulation of vascular smooth muscle cells plays an important role in the pathogenesis of arteriosclerosis. In a screen of proteins expressed in human aortic smooth muscle cells, e.g., as described in Example 1, we identified a novel gene product designated aortic carboxypeptidase-like protein (ACLP). The ˜4-kilobase human cDNA and its mouse homologue encode 1158 and 1128 amino acid proteins, respectively, that are 85% identical. ACLP is a nonnuclear protein that contains a signal peptide, a lysine- and proline-rich 11-amino acid repeating motif, a discoidin-like domain, and a C-terminal domain with 39% identity to carboxypeptidase E. By Western blot analysis and in situ hybridization, we detected abundant ACLP expression in the adult aorta. ACLP was expressed predominantly in the smooth muscle cells of the adult mouse aorta but not in the adventitia or in several other tissues. In cultured mouse aortic smooth muscle cells, ACLP mRNA and protein were up-regulated 2-3-fold after serum starvation. Using a recently developed neural crest cell to smooth muscle cell in vitro differentiation system, we found that ACLP mRNA and protein were not expressed in neural crest cells but were up-regulated dramatically with the differentiation of these cells. These results indicate that ACLP may play a role in differentiated vascular smooth muscle cells.

The origins of VSMCs during embryonic development are diverse (reviewed in Refs. 1, 3, and 4). During development, VSMCs derive from many cell types, such as local mesodermal precursors and neural crest cells (3, 5). Despite the fact that they express a similar set of smooth muscle cell marker genes, these cell populations can differ in morphology and respond in a lineage-dependent manner to factors such as transforming growth factor-1 (6). An understanding of the complex regulation of smooth muscle cell differentiation requires the identification of proteins involved in this response.

In a search for potential markers and regulators of smooth muscle cell growth and differentiation, we identified a novel gene product termed aortic carboxypeptidase-like protein (ACLP). ACLP contains a signal peptide, a repeating motif, a discoidin-like domain, and a domain with homology to the carboxypeptidases. ACLP is expressed highly in adult aortic smooth muscle cells, as detected by Northern blotting, Western blotting, and in situ hybridization. Also, expression of ACLP increases in cultured aortic smooth muscle cells after serum starvation. Using a recently developed in vitro system that allows the differentiation of multipotential mouse neural crest cells into smooth muscle cells, we show that ACLP is up-regulated dramatically. These results suggest that ACLP may play a role during development in the acquisition by VSMCs of the differentiated phenotype.

Experimental Procedures

Cell Lines, Cell Culture, and Reagents—Rat aortic smooth muscle cells (RASMCs) and mouse aortic smooth muscle cells (MASMCs) were isolated by the method of Gunther et al. (7) from the thoracic aortas of adult male Sprague-Dawley rats and C57B1/6 mice. Human aortic smooth muscle cells (HASMCs) were purchased from Clonetics (San Diego, Calif.), and rat A7r5 smooth muscle cells and C2C12 mouse myoblasts were purchased from the ATCC (Rockville, Md.). The mouse neural crest cell line Monc-1 was provided by David Anderson (Pasadena, Calif.). Monc-1 cells were cultured on fibronectin-coated plates as described (8), with minor modifications (9). RASMCs, MASMCs, and A7r5 cells were cultured in Dulbecco's modified Eagle's medium with 3.7 g/liter glucose (Life Technologies, Inc.) supplemented with 10% fetal bovine serum (Hyclone, Logan, Utah), 4 mM L-glutamine, 100 μg/ml streptomycin, 100 units/ml penicillin, and 10 mM HEPES (pH 7.4). C2C12 cells were grown in Dulbecco's modified Eagle's medium supplemented with 15% fetal bovine serum, 4 mM L-glutamine, 100 μg/ml streptomycin, and 100 units/ml penicillin. HASMCs were cultured in M199 medium (Life Technologies, Inc.) supplemented with 20% fetal bovine serum, 4 mM L-glutamine, 100 μg/ml streptomycin, and 100 units/ml penicillin. Cells were grown at 37° C. in a humidified incubator containing 5% CO2.

Cloning and Sequencing of Human and Mouse ACLP—A recombinant E47 fusion protein (N3-SH[ALA]) containing the basic helix loop helix domain of hamster shPan-1 (amino acids 509-646, with mutations R551A, V552L, and R553A) and a heart muscle kinase recognition sequence and FLAG epitope was expressed and purified as described (10, 11). The fusion protein was phosphorylated with heart muscle kinase in the presence of [-32P]ATP and then used to screen a human aorta gt11 cDNA expression library (1.5×106 pfu; CLONTECH, Palo Alto, Calif.) by interaction cloning (10, 11). A 1450-base pair (bp) cDNA clone that resulted from this interaction cloning was radiolabeled by random priming and used to isolate an ˜2.8-kilobase (kb) cDNA clone from the same human aorta gt11 cDNA library. Because Northern blotting revealed that the latter was also a partial cDNA clone, we isolated additional 5′ sequences from HASMC RNA by 5′ rapid amplification of cDNA ends (Life Technologies, Inc.).

GenBank™ searches revealed significant homology between the 3′ end of our human ACLP clone and mouse adipocyte enhancer-binding protein 1 (AEBP1) (12). To isolate the corresponding mouse ACLP cDNA, we synthesized first strand cDNA from C2C12 mouse myoblast total RNA by reverse transcription with the primer 5′-ATCTGGTTGTCCTCAAT-3′, which was designed according to the 5′ end of mouse AEBP1 (12). Using the nested primer 5′-TGACTCCATCCCAATAG-3′ and the anchor primer included in the kit for 5′ rapid amplification of cDNA ends, we amplified an ˜1400-bp fragment by the polymerase chain reaction (PCR). This product was ligated into pCR2.1 (Invitrogen, Carlsbad, Calif.) and sequenced as described below. The entire open reading frame of mouse ACLP was then amplified from C2C12 RNA by reverse transcription PCR (Expand Long Template PCR System, Boehringer Mannheim) and ligated into pCR2.1. We sequenced the human and the mouse clones by the dideoxy nucleotide chain termination method, using a combination of Sequenase Version 2.0 (Amersham Pharmacia Biotech), the Thermo Sequenase 33P terminator cycle sequencing kit (Amersham Pharmacia Biotech), and the Thermo Sequenase fluorescent-labeled cycle sequencing kit with 7-deaza-GTP (Amersham Pharmacia Biotech) on a Licor (Lincoln, Nebr.) apparatus.

Northern Blot Analysis—Total RNA was obtained from mouse organs by using RNAzol B according to manufacturer's instructions (Tel-Test, Inc., Friendswood, Tex.). RNA from cultured cells was isolated by guanidinium isothiocyanate extraction and centrifugation through cesium chloride (13). RNA was fractionated on 1.2% agarose (6% formaldehyde) gels and transferred to nitrocellulose filters (NitroPure, Micron Separations, Westboro, Mass.). The filters were hybridized with random-primed, 32P-labeled cDNA probes as described (13, 14). Equal loading was verified by hybridizing the filters to a 32P-labeled oligonucleotide complementary to 18S ribosomal RNA (15). Blots were exposed to x-ray film and a phosphor screen, and radioactivity was measured on a PhosphorImager running the ImageQuant software (Molecular Dynamics, Sunnyvale, Calif.) and normalized to 18S.

Cellular Localization of ACLP—To construct a c-myc-tagged ACLP expression plasmid (pcDNA3.1/ACLP-Myc-His), we amplified the open reading frame of mouse ACLP with the Expand Long Template PCR System (Boehringer Mannheim). We used a 5′ primer containing an EcoRI site (5′-CGGAATTCAGTCCCTGCTCAAGCCCG-3′) and a 3′ primer containing a HindIII site (5′-CGAAGCTTGAAGTCCCCAAAGTTCACTG-3′) to delete the endogenous termination codon. The PCR product was digested with EcoRI and HindIII restriction enzymes and ligated into the EcoRI and HindIII sites of pcDNA3.1( )/Myc-His A (Invitrogen). Cells were transiently transfected with pcDNA3.1/ACLP-Myc-His by the DEAE-dextran method with minor modifications (16). Twenty-four hours after transfection, cells were trypsinized, plated onto chamber slides (Nunc, Naperville, Ill.), and grown for an additional 24 h. Cells were fixed with 4% paraformaldehyde in phosphate-buffered saline and immunostained as described (17) with a monoclonal anti-c-myc primary antibody (9E10 Ab-1; Oncogene Research Products, Cambridge, Mass.) and a rhodamine-conjugated goat anti-mouse IgG secondary antibody. Nuclei were counterstained with Hoechst 33258 (1 μg/ml) and visualized with a fluorescence microscope.

Antibody Production and Western Blot Analysis—To produce a polyclonal anti-ACLP antibody, we subcloned a BamHI to EcoRI fragment of mouse ACLP (encoding amino acids 615-1128) into the pRSET C bacterial expression vector (Invitrogen). The plasmid was transformed into BL21(DE3)pLysS-competent bacteria (Stratagene), and protein expression was induced with 1 mM isopropyl-D-thiogalactopyranoside for 3 h. Bacteria were sonicated in lysis buffer (50 mM NaH2PO4, 10 mM Tris, pH 8, 100 mM NaCl) containing the protease inhibitors aprotinin, leupeptin, and phenylmethylsulfonyl fluoride. Lysates were clarified by centrifugation at 10,000× g for 15 min, and the pellet was resuspended in lysis buffer supplemented with 8 M urea. His-tagged proteins were purified with Talon resin (CLONTECH) and eluted in lysis buffer containing 8 M urea and 100 mM ethylene diamine tetraacetic acid. Proteins were dialyzed against water and measured with the Bio-Rad (Hercules, Calif.) protein assay reagent, and 100 μg was used to immunize New Zealand White rabbits. Antiserum was collected, titered against the recombinant protein, and used for immunoblot analysis as described below. Specificity of the antiserum was determined by using preimmune serum and by competition with a recombinant protein.

Protein extracts from cultured cells were prepared for Western blotting in extraction buffer (25 mM Tris, pH 7.4, 50 mM NaCl, 0.5% sodium deoxycholate, 2% Nonidet P-40, and 0.2% sodium dodecyl sulfate) containing the protease inhibitors aprotinin, leupeptin, and phenylmethylsulfonyl fluoride. To obtain proteins from mouse tissues, we homogenized individual organs in 25 mM Tris, pH 7.5, 50 mM NaCl, and 10 mM ethylene diamine tetraacetic acid containing protease inhibitors (Complete™, Boehringer Mannheim). Proteins were measured with the BCA protein assay kit (Pierce). After 50-μg aliquots had been resolved on 6% sodium dodecyl sulfate-polyacrylamide gels (18), proteins were transferred electrophoretically to nitrocellulose membranes (Schleicher and Schuell) in 48 mM Tris, pH 8.3, 39 mM glycine, 0.037% sodium dodecyl sulfate, and 20% methanol transfer buffer. Blots were equilibrated with 25 mM Tris, pH 8, 125 mM NaCl, and 0.1% Tween 20 and blocked in the same solution containing 4% nonfat dry milk. Blots were incubated with anti-ACLP serum diluted 1:1000 and then with horseradish peroxidase-conjugated goat anti-rabbit serum diluted 1:4000. Membranes were processed with an enhanced chemiluminescence reagent (NEN Life Science Products) and exposed to film.

In Situ Hybridization—Adult male Sprague-Dawley rats were perfused with 4% paraformaldehyde, and their organs were removed and sectioned (19). Probe was prepared, and in situ hybridization was conducted as described (19, 20). ACLP mRNA was detected with a [35S]UTP-labeled antisense riboprobe synthesized with SP6 RNA polymerase from a linearized 0.7-kb fragment of ACLP cDNA in pCR2.1. As a control, a sense RNA probe was synthesized with T7 RNA polymerase from a linearized ACLP cDNA fragment in pCR2.1.

Results

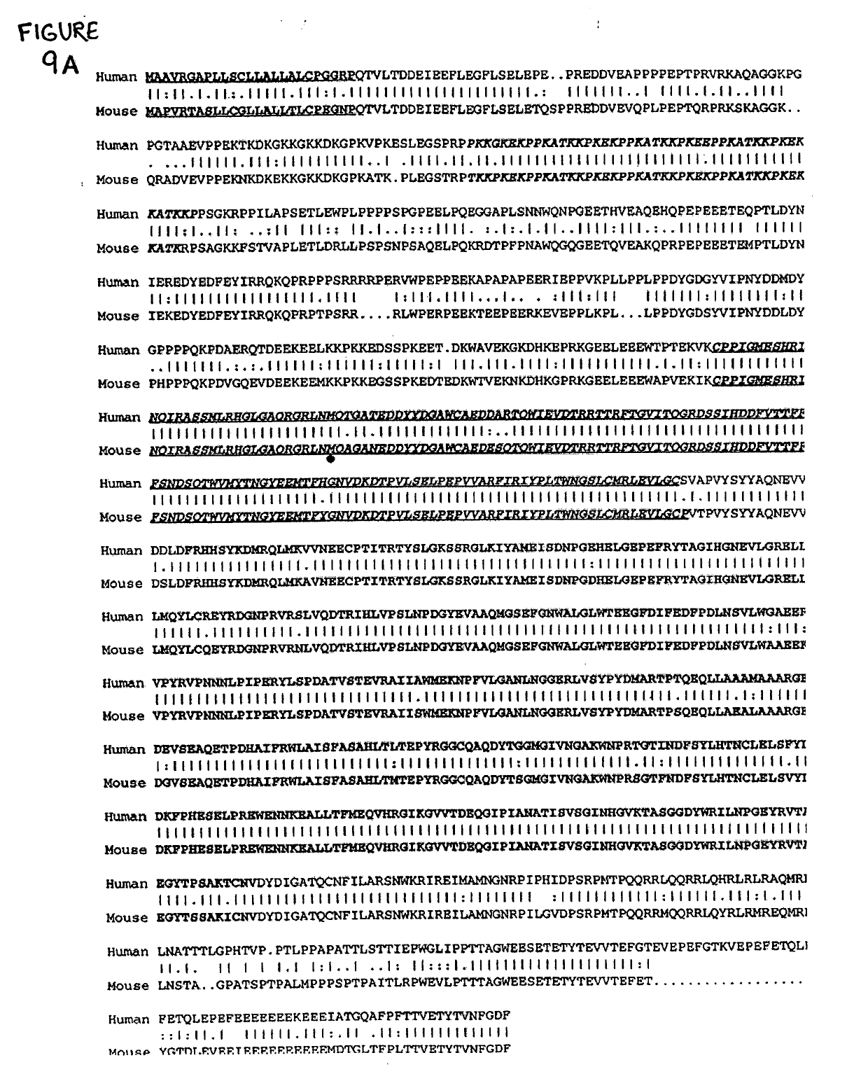

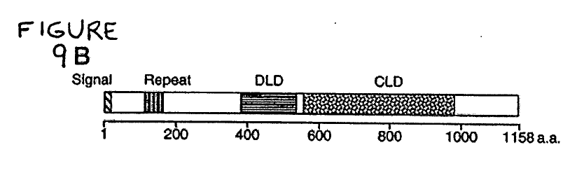

Isolation and Characterization of Human and Mouse ACLP cDNAs—To identify proteins interacting with products of the E2A gene (E12/E47) in VSMCs, we screened a human aorta expression library with a 32P-labeled E47 fusion protein. One truncated clone isolated from this screen (number 11) led to the full-length ACLP clone characterized here. Using in vitro binding assays, we determined that proteins derived from clone 11, but not from the full-length protein, bound to E12 and E47 (data not shown). The 3935 bp, full-length human ACLP cDNA contains an open reading frame of 1158 amino acids (FIG. 9A) and a Kozak consensus sequence for initiation of translation (GCCATGG) (21) preceded by an in-frame stop codon. The protein has a calculated molecular mass of 130 kDa and an estimated pI of 4.8, and it contains a putative signal peptide sequence (22, 23), an 11 amino acid lysine- and proline-rich motif repeated four times at the N terminus, a domain with 30% amino acid identity to the slime mold adhesion protein discoidin I, and a C-terminal domain with 39% identity to carboxypeptidase E (FIG. 9B).

GenBank™ searches revealed that the C terminus of human ACLP is highly homologous to mouse AEBP1 (12). AEBP1 was originally identified as an ˜2.5-kb cDNA that hybridized to an ˜4-kb band on Northern blot analysis, and it was predicted to encode a 719 amino acid, 79 kDa protein. The homology between ACLP and AEBP1 suggested two possibilities: either ACLP was a longer member of the AEBP1 gene family, or the AEBP1 sequence was substantially truncated at its 5′-end. To test the two possibilities, we cloned the mouse homologue of ACLP by a combination of 5′ rapid amplification of cDNA ends and reverse transcription PCR. After sequencing the 3633-bp mouse ACLP cDNA fragment, we found that it encoded an open reading frame (1128 amino acids) similar to that of our human clone, indicating that it is the mouse homologue (the two are 85% identical and 90% similar). Because AEBP1 is identical to the C terminus of the mouse ACLP, we conclude that the AEBP1 cDNA is probably not complete (the start of the AEBP1 sequence is indicated by a bullet in FIG. 9A).

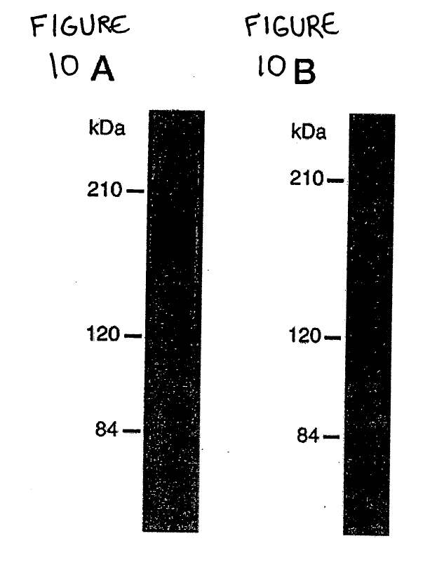

Characterization of ACLP—To confirm the putative open reading frame of mouse ACLP, we performed in vitro transcription and translation reactions with the mouse cDNA used as template. Translated products were resolved by sodium dodecyl sulfate-polyacrylamide gel electrophoresis, and a prominent band of ˜175 kDa was detected (FIG. 10A). To identify the endogenous ACLP, a C-terminal fragment of mouse ACLP was expressed in bacteria, purified, and used to raise antibodies in rabbits. By Western blot analysis, this antibody detected a single band with an apparent mobility of ˜175 kDa in MASMC extracts (FIG. 10B). The similar migration of the endogenous ACLP and the protein transcribed and translated in vitro indicates that we isolated a full-length cDNA clone.

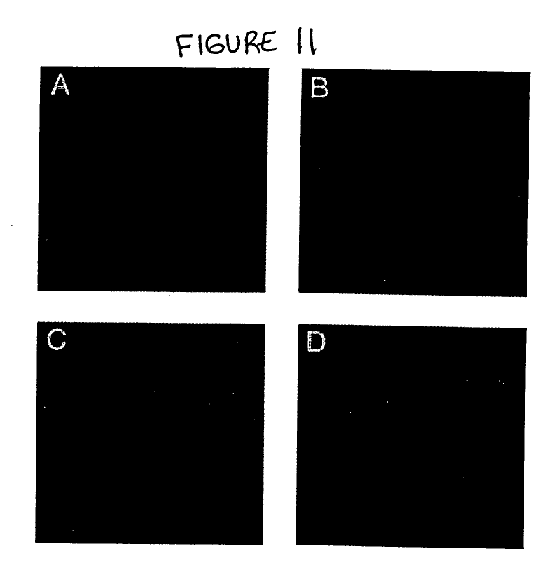

To assess the subcellular localization of ACLP, we generated a mouse ACLP expression construct with a c-myc epitope at the C terminus. The myc epitope was placed at the C terminus so that it would not interfere with signal peptide-mediated processes. This construct was transfected transiently into RASMCs and A7r5 cells, and immunostaining was performed with anti-c-myc antibody 9E10. RASMCs and A7r5 cells (FIG. 11, A and C) both exhibited strong membrane-associated or cytoplasmic staining. Staining was most intense in the perinuclear region and was not observed in the nucleus (FIG. 11, B and D).

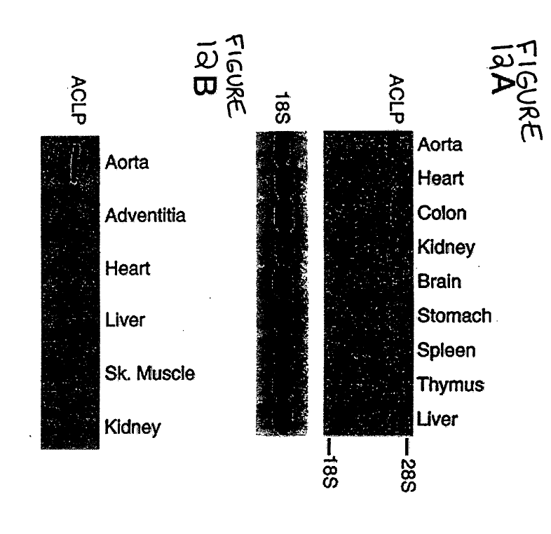

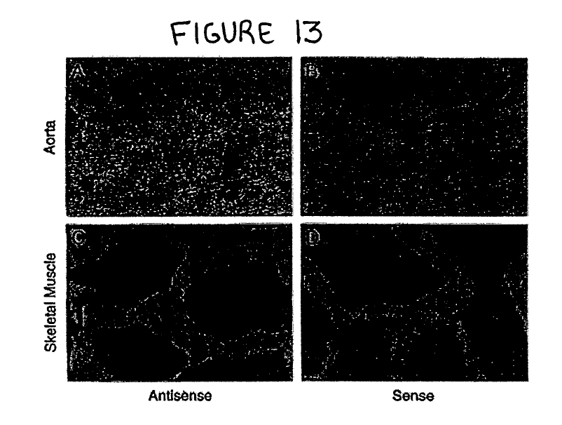

Tissue Expression of Mouse ACLP—Although the ACLP cDNA was cloned originally from aortic smooth muscle cells, we also wanted to examine its mRNA and protein expression in other tissues. As expected, levels of ACLP mRNA were high in the whole aorta (including adventitia) (FIG. 12A). Also, ACLP message was present in other tissues, including the colon and the kidney (FIG. 12A). To examine expression of ACLP, we subjected extracts from mouse tissues to Western blot analysis. ACLP was expressed abundantly in the mouse aorta (without adventitia) but not in the adventitia, heart, liver, skeletal muscle, or kidney (FIG. 12B). The presence of ACLP mRNA in the kidney (FIG. 12A) but absence of protein may indicate translational regulation. To identify cell types expressing ACLP in the adult, we performed in situ hybridization on adult rat aorta and skeletal muscle. The antisense riboprobe detected specific ACLP expression in the smooth muscle cells of the aorta (FIG. 13A), whereas the control, sense probe did not (FIG. 13B). As expected, neither the sense nor the antisense probe hybridized to skeletal muscle cells (FIG. 13, C and D).

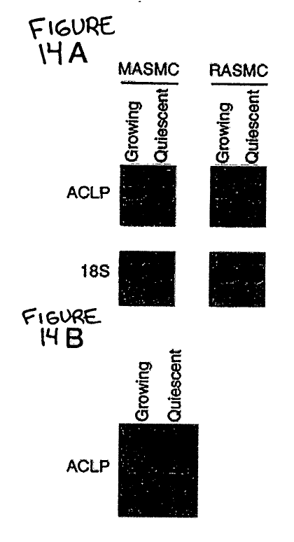

ACLP Expression in Cultured Smooth Muscle Cells—Because ACLP expression was high in the differentiated smooth muscle cells of the aorta (FIG. 11B), we examined the effect of VSMC growth and differentiation on ACLP expression. MASMCs were cultured for 3 days in 0.4% calf serum containing medium that induces quiescence. RNA and protein extracts were then prepared from the cells and analyzed. ACLP mRNA was more abundant (˜2-fold) in serum-starved MASMCs than in growing controls (FIG. 14A). In RASMCs, ACLP mRNA was ˜3-fold more abundant in quiescent cells than in their actively proliferating counterparts (FIG. 14A). ACLP was also elevated in quiescent MASMCs (FIG. 14B). Although these changes in message and protein levels are modest, they are consistent with increases in VSMC differentiation-specific markers observed in other systems (24, 25).

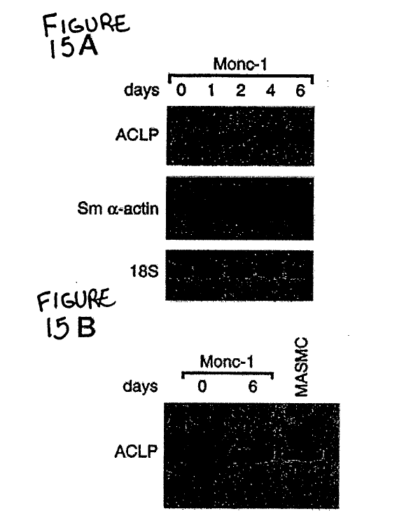

ACLP Expression in Smooth Muscle Cell Differentiation—Our laboratory recently developed an in vitro system for differentiating smooth muscle cells from Monc-1 cells, a mouse line derived from the neural crest (9). Monc-1 cells differentiate into smooth muscle cells when medium supplemented with chick embryo extract is replaced with differentiation medium (9). To examine ACLP expression during the conversion of undifferentiated Monc-1 cells to smooth muscle, we measured the time course-of ACLP expression. ACLP mRNA was nearly undetectable in undifferentiated Monc-1 cells (FIG. 15A). As the cells differentiated, however, ACLP expression increased until it became marked at days 4 and 6 after the start of differentiation (FIG. 15A). Under these conditions, induction of ACLP appeared to lag behind that of smooth muscle-actin, a marker for smooth muscle cells. To compare the level of ACLP in cells treated similarly, we prepared protein extracts from undifferentiated Monc-1 cells and cells allowed to differentiate for 6 days (FIG. 15B). ACLP was not detectable in undifferentiated Monc-1 cells (day 0) but was expressed highly (day 6) under conditions that promote Monc-1 cell differentiation into smooth muscle cells. The abundance of ACLP in these cells was similar to that in MASMCs.

This example describes the cloning of a novel cDNA from human aortic smooth muscle cells, termed ACLP, and its mouse homologue. Notable features of the protein include a predicted signal peptide sequence at the N terminus, a lysine- and proline-rich 11-amino acid repeat, a discoidin-like domain, and a large C-terminal carboxypeptidase-like domain (FIG. 9B).

The screen that led to the identification of ACLP was performed to identify binding partners of the E2A proteins. The products of the E2A gene, E12 and E47, serve as heterodimerization partners for tissue specific transcription factors that regulate growth and differentiation in several cell types. Although the E2A gene products are expressed ubiquitously (26), a vascular smooth muscle specific heterodimerization partner or transcription factor has not been identified. We cloned the C-terminal portion of human ACLP (amino acids 793-1158) by using a labeled E47 protein probe and verified its binding to E47 by in vitro assays (data not shown). However, the full-length ACLP, because of its predicted signal peptide sequence (FIG. 9) and nonnuclear subcellular localization (FIG. 11), probably does not function as a heterodimerization partner for E47 in vivo.

GenBank™ searches indicated high homology between the C terminus of human ACLP and the mouse AEBP1 described by He et al. (12). To determine the relation between ACLP and AEBP1, we cloned the mouse ACLP cDNA. By sequence comparison, AEBP1 was found to be identical to mouse ACLP, beginning at ACLP methionine 410 (FIG. 9A). We then determined that ACLP is a single-copy gene in the mouse and cloned the region corresponding to the 5′ end of AEBP1 from genomic DNA.2 Analysis of the genomic clone confirmed that the AEBP1 sequence is missing a G residue 11 bases 5′ to the identified ATG. The presence of this G residue in ACLP would eliminate the in frame stop codon proposed by He et al. (12) and extend the open reading frame.

The 2.5-kb AEBP1 cDNA is unlikely to code for an authentic protein. Probes derived from AEBP1 and both the 5′ and 3′ ends of ACLP detected a single, ˜4-kb band by Northern blot analysis, which is consistent with the size of the human as well as the mouse ACLP cloned cDNAs. Because the AEBP1 cDNA contains a putative polyadenylation signal and a poly(A) tail, the difference between the AEBP1 cDNA and mRNA is ˜1.5 kb. This missing 1.5 kb of sequence is present in the 5′ end of the ACLP cDNA. Also, the anti-ACLP antibody generated for these studies was raised from the C terminus of ACLP, which is identical to AEBP1. The antibody detected only a single band of ˜175 kDa by Western blotting in several tissues examined (FIG. 12B), which is consistent with the mobility of ACLP transcribed and translated in vitro (FIG. 10). We also detected a single band of identical mobility in protein extracts from several cell lines in culture, including 3T3-L1 preadipocytes. ACLP was expressed in 3T3-L1 preadipocytes at substantially lower levels than in MASMCs or differentiated Monc-1 cells (data not shown). Thus, AEBP1 appears to be a truncated clone of mouse ACLP. AEBP1 is missing the ACLP signal peptide, repeat domain, and part of the discoidin domain.

ACLP has a prominent carboxypeptidase-like domain of about 500 amino acids at its C terminus (FIG. 9B). This domain is 39% identical to carboxypeptidase E. Despite this high sequence similarity, however, we3 and others (27) have been unable to demonstrate that this domain of ACLP has any catalytic carboxypeptidase activity. These results may reflect the divergence of specific residues in ACLP from sequences of the carboxypeptidase family (27). For example, a histidine involved in zinc binding in carboxypeptidases is replaced by an asparagine (amino acid 763) in human ACLP. Catalytically important tyrosine and glutamic acid residues in the carboxypeptidases are substituted by asparagine (amino acid 852) and tyrosine (amino acid 874) in human ACLP, respectively. Also, the positively charged arginine residue in the substrate recognition pocket of the carboxypeptidases that stabilizes the C-terminal carboxyl group of the substrate is replaced by a negatively charged glutamic acid residue (amino acid 700) in human ACLP. Although catalytically inactive, ACLP may interact with other proteins via this carboxypeptidase-like domain, as evidenced by our initial isolation of the ACLP cDNA from an expression library screened with a 32P-labeled protein probe. Carboxypeptidase E also serves as a sorting receptor in the secretory pathway (28), implicating functions other than catalysis for the carboxypeptidase domain.

The second important motif in ACLP is a discoidin-like domain (FIG. 9B), which has been identified in coagulation factors V and VIII (29-31), milk fat globule membrane proteins (32, 33), the discoidin domain tyrosine kinase receptor (34), the endothelial cell protein del-1 (35), and the A5/neuropilin protein (36-38). Discoidin is a lectin produced by the slime mold Dictyostelium discoideum and is thought to facilitate cellular aggregation and migration by functioning as fibronectin does in vertebrates (39). ACLP and many other proteins containing a discoidin-like domain lack the RGD motif important to the function of both discoidin and fibronectin (40). The discoidin-like domain may be important for cell-cell recognition, or it may be involved in cell migration mediated through homotypic and heterotypic interactions (36, 39). The discoidin domain tyrosine kinase receptors are activated by collagen, although the receptor domain involved in this interaction has not been identified (41, 42). The discoidin-like domain may also bind to phospholipids (33, 43). As ACLP lacks a predicted transmembrane-spanning domain (FIG. 9A), the discoidin-like domain may mediate the interaction of ACLP with the cell membrane.

Although ACLP is not expressed in neural crest cells, it is induced markedly during Monc-1 cell to smooth muscle cell differentiation (FIG. 15). This induction of ACLP during Monc-1 differentiation, in conjunction with the preferential expression of ACLP in VSMCs in vivo (FIG. 12), links ACLP expression to the development of the VSMC lineage. Moreover, induction of ACLP by culture medium that confers a differentiated VSMC phenotype (FIG. 14) further suggests a role for ACLP in the differentiation of this cell type.

The abbreviations used are: VSMC, vascular smooth muscle cell; ACLP, aortic carboxypeptidase-like protein; RASMC, rat aortic smooth muscle cell; MASMC, mouse aortic smooth muscle cell; HASMC, human aortic smooth muscle cell; bp, base pair(s); kb, kilobase(s); AEBP1, adipocyte enhancer-binding protein 1; PCR, polymerase chain reaction.

### REFERENCES FOR EXAMPLE 2

1. Owens, G. K. (1995) Physiol. Rev. 75, 487-517 2. Ross, R. (1993) Nature 362, 801-809 3. Kirby, M. L., and Waldo, K. L. (1995) Circ. Res. 77,211-215 4. Kirby, M. L., and Waldo, K. L. (1990) Circulation 82, 332-340 5. Noden, D. M. (1989) Am. Rev. Respir. Dis. 140, 1097-1103 6. Topouzis, S., and Majesky, M. W. (1996) Dev. Biol. 178, 430-445 7. Gunther, S., Alexander, R. W., Atkinson, W. J., and Gimbrone, M. A., Jr. (1982) J. Cell. Biol. 92, 289-298 8. Stemple, D. L., and Anderson, D. J. (1992) Cell 71, 973-985 9. Jain, M. K., Layne, M. D., Watanabe, M., Chin, M. T., Feinberg, M. W., Sibinga, N. E. S., Hsieh, C.-M., Yet, S.-F., Stemple, D. L., and Lee, M.-E. (1998) J. Biol. Chem. 273, 5993-5996 10. Blanar, M. A., and Rutter, W. J. (1992) Science 256, 1014-1018 11. Blanar, M. A., Crossley, P. H., Peters, K. G., Steingrimsson, E., Copeland, N. G., Jenkins, N. A., Martin, G. R., and Rutter, W. J. (1995) Proc. Natl. Acad. Sci. U. S. A. 92, 5870-5874 12. He, G.-P., Muise, A., Li, A. W., and Ro, H.-S. (1995) Nature 378, 92-96 13. Sambrook, J., Fritsch, E. F., and Maniatis, T. (1989) Molecular Cloning: A Laboratory Manual, Cold Spring Harbor Laboratory, Cold Spring Harbor, N.Y. 14. Yoshizumi, M., Lee, W.-S., Hsieh, C.-M., Tsai, J.-C., Li, J., Perrella, M. A., Patterson, C., Endege, W. O., Schlegel, R., and Lee, M.-E. (1995) J. Clin. Invest. 95, 2275-2280 15. Yoshizumi, M., Kourembanas, S., Temizer, D. H., Cambria, R. P., Quertermous, T., and Lee, M.-E. (1992) J. Biol. Chem. 267, 9467-9469 16. Tan, M., Tsai, J. T., Lee, Y. J., Chen, H. C., Shin, S. J., Lai, Y. H., Perrella, M. A., Bianchi, C., Higashiyama, S., Endege, W., Lee, M. E., and Tsai, J. H. (1994) Kidney Int. 46, 690-695 17. Hsieh, C.-M., Yoshizumi, M., Endege, W. O., Kho, C.-J., Jain, M. K., Kashiki, S., de los Santos, R., Lee, W.-S., Perrella, M. A., and Lee, M.-E. (1996) J. Biol. Chem. 271, 17354-17359 18. Laemmli, U. K. (1970) Nature 227, 680-685 19. Lee, W.-S., Berry, M. J., Hediger, M. A., and Larsen, P. R. (1993) Endocrinology 132, 2136-2140 20. Lee, W.-S., Kanai, Y., Wells, R. G., and Hediger, M. A. (1994) J. Biol. Chem. 269, 12032-12039 21. Kozak, M. (1992) Annu. Rev. Cell Biol. 8, 197-225 22. von Heijne, G. (1985) J. Mol. Biol. 184, 99-105 23. Brendel, V., Bucher, P., Nourbakhsh, I. R., Blaisdell, B. E., and Karlin, S. (1992) Proc. Natl. Acad. Sci. U. S. A. 89, 2002-2006 24. Owens, G. K., Loeb, A., Gordon, D., and Thompson, M. M. (1986) J. Cell. Biol. 102, 343-352 25. Li, X., Tsai, P., Weider, E. D., Kribben, A., Van Putten, V., Schrier, R. W., and Nemenoff, R. A. (1994) J. Biol. Chem. 269, 19653-19658 26. Roberts, V. J., Steenbergen, R. S., and Murre, C. (1993) Proc. Natl. Acad. Sci U. S. A. 90, 7583-7587 27. Song, L., and Fricker, L. D. (1997) J. Biol. Chem. 272, 10543-10550 28. Cool, D. R., Normant, E., Shen, F., Chen, H.-C., Pannell, L., Zhang, Y., and Loh, Y. P. (1997) Cell 88, 73-83 29. Toole, J. J., Knopf, J. L., Wozney, J. M., Sultzman, L. A., Buecker, J. L., Pittman, D. D., Kaufman, R. J., Brown, E., Shoemaker, C., Orr, E. C., Amphlett, G. W., Foster, W. B., Coe, M. L., Knutson, G. J., Fass, D. N., and Hewick, R. M. (1984) Nature 312, 342-347 30. Kane, W. H., and Davie, E. W. (1986).Proc. Natl. Acad. Sci. U. S. A. 83, 6800-6804 31. Kane, W. H., and Davie, E. W. (1988) Blood 71, 539-555 32. Stubbs, J. D., Lekutis, C., Singer, K. L., Bui, A., Yuzuki, D., Srinivasan, U., and Parry, G. (1990) Proc. Natl. Acad. Sci. U. S. A. 87, 8417-8421 33. Larocca, D., Peterson, J. A., Urrea, R., Kuniyoshi, J., Bistrain, A. M., and Ceriani, R. L. (1991) Cancer Res. 51, 4994-4998 34. Johnson, J. D., Edman, J. C., and Rutter, W. J. (1993) Proc. Natl. Acad. Sci. U. S. A. 90, 5677-5681 35. Hidai, C., Zupancic, T., Penta, K., Mikhail, A., Kawana, M., Quertermous, E. E., Aoka, Y., Fukagawa, M., Matsui, Y., Platika, D., Auerbach, R., Hogan, B. L. M., Snodgrass, R., and Quertermous, T. (1998) Genes Dev. 12, 21-33 36. Takagi, S., Hirata, T., Agata, K., Mochii, M., Eguchi, G., and Fujisawa, H. (1991) Neuron 7, 295-307 37. He, Z., and Tessier-Lavigne, M. (1997) Cell 90, 739-751 38. Kolodkin, A. L., Levengood, D. V., Rowe, E. G., Tai, Y.-T., Giger, R. J., and Ginty, D. G. (1997) Cell 90, 753-762 39. Springer, W. R., Cooper, D. N. W., and Barondes, S. H. (1984) Cell 39, 557-564 40. Pierschbacher, M. D., Hayman, E. G., and Ruoslahti, E. (1985) J. Cell. Biochem. 28, 115-126 41. Shrivastava, A., Radziejewski, C., Campbell, E., Kovac, L., McGlynn, M., Ryan, T. E., Davis, S., Goldfarb, M. P., Glass, D. J., Lemke, G., and Yancopoulos, G. D. (1998) Mol. Cell 1, 25-34 42. Vogel, W., Gish, G. D., Alves, F., and Pawson, T. (1998) Mol. Cell 1, 13-23 43. Tracy, P. B., and Mann, K. G. (1983) Proc. Natl. Acad. Sci. U. S. A. 80, 2380-2384

All of the above-cited references and publications are hereby incorporated by reference.

Equivalents

Those skilled in the art will recognize, or be able to ascertain using no more than routine experimentation, numerous equivalents to the specific method and reagents described herein. Such equivalents are considered to be within the scope of this invention and are covered by the following claims.

What is claimed:

1. A method for stimulating the differentiation of vascular smooth muscle cells comprising culturing neural crest cells under conditions wherein SM22α gene expression is induced.

2. The method of claim 1, wherien the neural crest cells are immortalized cells.

3. The method of claim 1, wherein the neural crest cells are cultured in smooth muscle cell differentiation medium.

4. The method of claim 3, wherein said smooth muscle cell differentiation medium comprises the media components listed in Table 1 supplemented with 10% fetal bovine serum, penicillin (100 units/ml), streptomycin (100 μg/ml), and 25 mM Hepes (pH 7.4).

5. The method of claim 3, wherein said immortalized neural crest cells are first cultured in complete medium.

6. The method of claim 5, wherein said complete medium comprises an L-15 CO₂-based medium supplemented with chick embryo extract.

7. A method for identifying a gene which regulates proliferation or migration of a smooth muscle cells, comprising
(i) culturing neural crest cells under culture conditions wherein SM22α: gene expression is induced for a time period sufficient for the neural crest cells to begin differentiation to smooth muscle cells;
(ii) identifying genes which are up- or down-regulated under the culture conditions.

8. The method of claim 7, wherein the step of identifying genes which are up- or down-regulated includes differential display of mRNA from the culture cells with mRNA from non-smooth muscle cells.

9. The method of claim 7, wherein one or more genes which are up- or down-regulated under the culture conditions are cloned.

10. A method of identifying an agent that modulates which regulates proliferation or migration of smooth muscle cells, comprising:
(a) stimulating the differentiation of vascular smooth muscle cells comprising culturing neural crest cells under culture conditions wherein SM22α gene expression is induced for a time period sufficient for the neural crest cells to begin differentiation to smooth muscle cells;
(b) contacting the cells with a test agent; and
(c) measuring the ability of the test agent to inhibit the differentiation of the neural crest cells to smooth muscle cells.

11. The method of claim 10, wherein the ability of the agent to inhibit the differentiation of the neural crest cells to smooth muscle cells is measured by detecting the presence or absence of smooth muscle cell markers.

12. A method for identifying an agent that modulates proliferation or migration of smooth muscle cells, comprising:
(i) identifying a gene product which is up- or down-regulated during differentiation of neural crest cells to smooth muscle cells;
(ii) identifying an agent which inhibits or potentiates an activity of the gene product.

13. The method of claim 12, wherein the gene product is selected from the group consisting of a latent TGFβ binding protein, an integrin-linked kinase, an aortic carboxypeptidase, a a Torsin, cctζ, a prothymosin, Limk2, Cca (confluent 3Y1 cell-associated), an interferon activatible protein, an intemexin, a Caspase, AHNAK, Desmoyokin, TSC-36 (TGF inducible protein), Transcobalamin, a fos-related antigen, an epididymal secretory protein E1 precursor (HE1), a ubiquitin carboxyl-terminal hydrolase, a thyrptropin releasing hormone, and a Decorin.

14. A method for identifying an agent that inhibits proliferation and/or migration of smooth muscle cells, comprising:
(i) identifying a gene product which is up-regulated during differentiation of neural crest cells to smooth muscle cells, the gene product having a biological activity required for proliferation and/or migration of smooth muscle cells;
(ii) identifying an agent which inhibits the biological activity of the gene product.

15. A method for identifying an agent that modulates proliferation and/or migration of smooth muscle cells, comprising:
(i) identifying an agent which alters the biological activity of a latent TGFβ binding protein (LTBP), and
(ii) assessing the ability of the agent to modulate proliferation and/or migration of smooth muscle cells.

16. The method of claim 15, wherein the agent inhibits interaction of the LTBP with a TGFβ complex.

17. The method of claim 14, wherein the agent inhibits proteolytic cleavage of LTBP.

18. A method for identifying an agent that modulates proliferation and/or migration of smooth muscle cells, comprising:
(i) identifying an agent which inhibits the kinase activity of an integrin linked kinase, and
(ii) assessing the ability of the agent to modulate proliferation and/or migration of smooth muscle cells.

19. The method of claim 15, wherein the agent inhibits phosphorylation of integrin subunits.

20. A method for identifying an agent that modulates proliferation and/or migration of smooth muscle cells, comprising:
(i) identifying an agent which inhibits activation of a LEF-1/β-catenin signaling pathway, and
(ii) assessing the ability of the agent to modulate proliferation and/or migration of smooth muscle cells.

21. The method of any of claims 10, 12, 14, 15, 18 or 20, comprising the further step of formulating a pharmaceutical preparation comprising one or more agents identified as able to modulate proliferation and/or migration of smooth muscle cells.

22. A method for modulating proliferation and/or migration of smooth muscle cells comprising contacting the smooth muscle cells with an agent identified by the method of any of claims 10, 12, 14, 15, 18 or 20 as able to modulate proliferation and/or migration of smooth muscle cells.

23. The method of claim 22, wherein the smooth muscle cells are contacted with the agent in vitro.

24. The method of claim 22, wherein the agent is administered to animal in order to treat or prevent unwanted proliferation of smooth muscle cells.

25. The method of claim 24, wherein the agent is administered to animal in order to treat or prevent restenosis.

26. The method of claim 24, wherein the agent is administered to animal in order to treat or prevent atherosclerosis.

27. The method of claim 24, wherein the agent is administered to animal in order to maintain an expanded luminal volume following angioplasty or other vessel trauma.

28. A method for treating or preventing unwanted proliferation of smooth muscle cells in animal, comprising:
(i) identifying an agent which inhibits the biological activity of, or inhibits expression of, a gene product which is up-regulated during differentiation of neural crest cells to smooth muscle cells, the gene product having a biological activity required for proliferation and/or migration of smooth muscle cells; and
(ii) administering to animal in need thereof an amount of the agent which is effective to inhibit unwanted proliferation of smooth muscle cells.

29. A method for treating or preventing abnormal, pathological or inappropriate proliferation of smooth muscle cells in animal, comprising:
(i) identifying a gene product which is up-regulated during differentiation of neural crest cells to smooth muscle cells, the gene product having a biological activity required for proliferation and/or migration of smooth muscle cells;
(ii) identifying an agent which inhibits the biological activity of the gene product, or which inhibits expression of the gene product, so as to inhibit proliferation of smooth muscle cells; and
(iii) administering to animal in need thereof an amount of the agent which is effective to inhibit unwanted proliferation of smooth muscle cells.

30. A method for identifying an agent that inhibits proliferation of smooth muscle cells, comprising:
(i) identifying an agent which inhibits the biological activity of a gene product which is up-regulated during differentiation of neural crest cells to smooth muscle cells, the gene product having a biological activity required for proliferation and/or migration of smooth muscle cells; and
(ii) assessing the ability of the agent, or an analog thereof, to modulate proliferation and/or migration of smooth muscle cells.


SEQUENCE LISTING (23 sequences)

SEQ ID NO: 1 - DNA, 153 bp, Murine
  Clone WO11
agtggtgtatgtggggaagccttctgtgaaaacgtggaaggatccttcctgtgtgtgtgtgccgatgagaaccaggagtacagccccatgactgggcagtgtcgctctcgggtcacggaagattcaggtgtggatcgtcagcctaggcgaaga

SEQ ID NO: 2 - DNA, 143 bp, Rattus
  Clone LTBP-1
agtggtgtatgtggggaggctttctgtgaaaatgtggaagggtccttcctgtgcgtgtgtgccgatgagaaccaggagtacagccccatgactgggcagtgtcgctcccgggctactgaagattcaggtgtggatcgtcagcc

SEQ ID NO: 3 - protein, 1128 aa, Homo sapiens [sequence omitted]

SEQ ID NO: 4 - protein, 1102 aa, Murine
  ACLP
MAPVRTASLLCGLLALLTLCPEGNPQTVLTDDEIEEFLEGFLSELETQSPPREDDVEVQPLPEPTQRPRKSKAGGKQRADVEVPPEKNKDKEKKGKKDKGPKATKPLEGSTRPTKKPKEKPPKATKKPKEKPPKATKKPKEKPPKATKKPKEKKATKRPSAGKKFSTVAPLETLDRLLPSPSNPSAQELPQKRDTPFPNAWQGQGEETQVEAKQPRPEPEEETEMPTLDYNIEKEDYEDFEYIRRQKQPRPTPSRRRLWPERPEEKTEEPEERKEVEPPLKPLLPPDYGDSYVIPNYDDLDYPHPPPQKPDVGQEVDEEKEEMKKPKKEGSSPKEDTEDKWTVEKNKDHKGPRKGEELEEEWAPVEKIKCPPIGMESHRINQIRASSMLRHGLGAQRGRLNMQAGANEDDYYDGAWCAEDESQTQWIEVDTRRTTRFTGVITQGRDSSIHDDFVTTFFFSNDSQTWVMYTNGYEEMTFYGNVDKDTPVLSELPEPVVARFIRIYPLTWNGSLCMRLEVLGCPVTPVYSYYAQNEVVDSLDFRHHSYKDMRQLMKAVNEECPTITRTYSLGKSSRGLKIYAMEISDNPGDHELGEPEFRYTAGIHGNEVLGRELILMQYLCQEYRDGNPRVRNLVQDTRIHLVPSLNPDGYEVAAQMGSEFGNWALGLWTEEGFDIFEDFPDLNSVLWAAEEFVPYRVPNNNLPIPERYLSPDATVSTEVRAIISWMEKNPFVLGANLNGGERLVSYPYDMARTPSQEQLLAEALAAAEGEDGVSEAQETPDHAIFRWLAISFASAHLTMTEPYRGGCQAQDYTSGMGIVNGAKWNPRSGTFNDFSYLHTNCLELSVYIDKFPHESELPREWENNKEALLTFMEQVHRGIKGVVTDEQGIPIANATISVSGINHGVKTASGGDYWRILNPGEYRVTAEGYTSSAKICNVDYDIGATQCNFILARSNWKRIREILAMNGNRPILGVDPSRPMTPQQRRMQQRRLQYRLRMREQMRLNSTAGPATSPTPALMPPPSPTPAITLRPWEVLPTTTAGWEESETETYTEVVTEFETVFTDLEVEELEEEEEREEEEMDTGLTFPLTTVETYTVNFGDF

SEQ ID NO: 5 - DNA, 10 bp, Artificial Sequence
  Description of Artificial Sequence CArG box
ccwwwwwwgg

SEQ ID NO: 6 - DNA, 30 bp, Artificial Sequence
  Description of Artificial Sequence CArG box (3X)
ccwwwwwwggccwwwwwwggccwwwwwwgg

SEQ ID NO: 7 - DNA, 10 bp, Artificial Sequence
  Description of Artificial Sequence modified oligo
ccaaatatgg

SEQ ID NO: 8 - DNA, 10 bp, Artificial Sequence
  Description of Artificial Sequence modified oligo
ccacacatgg

SEQ ID NO: 9 - DNA, 29 bp, Artificial Sequence
  Description of Artificial Sequence primer
acgactcactatagggcttttttttttgt

SEQ ID NO: 10 - DNA, 26 bp, Artificial Sequence
  Description of Artificial Sequence primer
acaatttcacacaggatggtaaaccc

SEQ ID NO: 11 - DNA, 21 bp, Artificial Sequence
  Description of Artificial Sequence primer
agccaaggagcccaccaaact

SEQ ID NO: 12 - DNA, 21 bp, Artificial Sequence
  Description of Artificial Sequence primer
ttaccacgatgttcctcttga

SEQ ID NO: 13 - DNA, 21 bp, Artificial Sequence
  Description of Artificial Sequence primer
aggaaacaccaaggtcaagca

SEQ ID NO: 14 - DNA, 20 bp, Artificial Sequence
  Description of Artificial Sequence primer
gggactgtaccacaggttag

SEQ ID NO: 15 - DNA, 26 bp, Artificial Sequence
  Description of Artificial Sequence primer
tgaaggtcggtgtgaacggatttggc

SEQ ID NO: 16 - DNA, 24 bp, Artificial Sequence
  Description of Artificial Sequence primer
catgtaggccatgaggtccaccac

SEQ ID NO: 17 - DNA, 21 bp, Artificial Sequence
  Description of Artificial Sequence primer
cagtggctggaaagcaagagc

SEQ ID NO: 18 - DNA, 18 bp, Artificial Sequence
  Description of Artificial Sequence primer
gggctggggcagacgggc

SEQ ID NO: 19 - DNA, 45 bp, Artificial Sequence
  Description of Artificial Sequence CArG element
tcgagacttggtgtctttccccaaatatggagcctgtgtggagtg

SEQ ID NO: 20 - DNA, 17 bp, Artificial Sequence
  Description of Artificial Sequence primer
atctggttgtcctcaat

SEQ ID NO: 21 - DNA, 17 bp, Artificial Sequence
  Description of Artificial Sequence primer
tgactccatcccaatag

SEQ ID NO: 22 - DNA, 26 bp, Artificial Sequence
  Description of Artificial Sequence EcoRI site
cggaattcagtccctgctcaagcccg

SEQ ID NO: 23 - DNA, 28 bp, Artificial Sequence
  Description of Artificial Sequence HindIII site
cgaagcttgaagtccccaaagttcactg